(19) United States
(12) Patent Application Publication
(10) Pub. No.: US 2021/0260280 A1
(43) Pub. Date: Aug. 26, 2021

(54) MAGNETIC RESONANCE IMAGING COMPATIBLE CONVECTION-ENHANCED DELIVERY CRANIAL IMPLANT DEVICES AND RELATED METHODS

(71) Applicant: THE JOHNS HOPKINS UNIVERSITY, Baltimore, MD (US)

(72) Inventors: Chad R. Gordon, Cockeysville, MD (US); Jon D. Weingart, Phoenix, MD (US); SeYeon Choi, Baltimore, MD (US); Victor Dadfar, Baltimore, MD (US); Victoria Fang, Baltimore, MD (US); Daniel Huang, Baltimore, MD (US); Zhou Li, Baltimore, MD (US); Angela Lu, Baltimore, MD (US); Hadley Van-Renterghem, Baltimore, MD (US); Deborah Weidman, Baltimore, MD (US)

(21) Appl. No.: 17/256,711

(22) PCT Filed: Jun. 27, 2019
(86) PCT No.: PCT/US2019/039519

§ 371 Date: Dec. 29, 2020

Related U.S. Application Data
(60) Provisional application No. 62/692,111, filed on Jun. 29, 2018.

(51) Int. Cl. A61M 5/142; A61F 2/28

(52) U.S. Cl. CPC A61M 5/14276; A61F 2002/3009; A61M 2210/0693; A61F 2/2875

(57) ABSTRACT

Provided herein are magnetic resonance imaging (MRI) compatible, convection-enhanced delivery (CED) cranial implant devices and related methods for performing a wide array of therapeutic and/or monitoring applications. In one aspect, the cranial implant device includes a cranial implant housing configured for intercranial implantation in a cranial opening of a subject. The cranial implant housing comprises a substantially anatomically-compatible shape, at least first and second surfaces, and at least one fluidic circuit comprising at least one cavity and at least one port that fluidly communicates with the cavity through at least the second surface, in which the cavity comprises, or is capable of comprising, at least one fluidic therapeutic agent. The device also includes at least one CED pump operably connected to the fluidic circuit, which CED pump is configured to convey the fluidic therapeutic agent from the cavity through at least one fluid conduit when the fluid conduit is operably connected to the port to maintain at least one positive pressure gradient of the fluidic therapeutic agent at least proximal to an outlet of the fluid conduit. In addition, the device also includes at least one power source operably connected at least to the CED pump. The cranial implant housing, the CED pump, and the power source are typically fabricated from one or more MRI compatible materials. Other aspects relate to various methods of treating a neurologically-related disease using the cranial implant devices, methods of monitoring therapeutic agent administration in a plurality of subjects, and methods of fabricating a cranial implant device as well as surgical methods.

### CROSS-REFERENCE TO RELATED APPLICATIONS

This application claims priority to U.S. Provisional Patent Application Ser. No. 62/692,111, filed Jun. 29, 2018, the entirety of which is incorporated herein by reference.

### BACKGROUND

Challenges surrounding the blood-brain barrier and common neurological diseases, like malignant brain tumors for example, have remained daunting to neurosurgeons and neuro-oncologists alike (Vogelbaum et al., “Convection-enhanced delivery for the treatment of glioblastoma,” Neuro Oncol., 17(2):3-8 (2015)). In parallel with these challenges, optimization of cranial implant size and dimension is needed to ensure optimal reconstruction with absent visual deformity and biocompatible placement to avoid impinging the scalp from underneath or the brain from above, which assures safer outcomes for patients in need of cranial implant or cranioplasty reconstruction. Recent innovations in implant design, mainly those that emolliate the issue of temporal hollowing following wasting of the temporalis muscle and temporal fat pad, have made great strides by adding additional thickness to the standard size pterional cranial implant (Zhong et al., “Quantitative analysis of dual-purpose, patient-specific craniofacial implants for correction of temporal deformity,” Neurosurgery, 11:220-229 (2015) and U.S. Patent Application Publication No. US 2019/0021863) which in turn, provides room for embedded technologies.

Neuro-oncologists, neurosurgeons, and neuroplastic surgeons are all in need of a chronic method to deliver therapeutics directly to recurrent glioblastoma multiforme tumor sites in order to extend patient life. As such, convection-enhanced delivery (CED) is a direct (i.e., local) medicine delivery technique that has shown great promise for an otherwise challenging dilemma with respect to brain tumors and effective chemotherapy delivery. In summary, the CED technique in pre-existing form involves connecting the patient's head directly to a tall intravenous pole with pressure-assisted flow to overcome resistance via several brain catheters placed through the scalp and small skull defects, and kept in place for just 5-10 days maximum due to the risk of infection. This pre-existing approach is able to generate a pressure gradient at the tip of an infusion catheter to deliver therapeutics directly through the interstitial spaces of the central nervous system (CNS), which has suggested improved survival with respect to standard chemotherapy for malignant brain tumors like glioblastoma by avoiding toxic metabolites observed with therapies like radiation and/or intravenous/oral chemotherapy. However, the limit of CED's pre-existing applicability is the fact that there is no reliable delivery vehicle to allow CED to occur past 5-10 days, for example, in a way that would be chronic, safe, effective, allow the patient to be discharged from the hospital, and could avoid any form of visible deformity with the accompanying social stigmata of being treated for brain cancer.

### SUMMARY

This application discloses magnetic resonance imaging (MRI) compatible, convection-enhanced delivery (CED) cranial implant devices and related methods for performing a wide array of therapeutic and/or monitoring applications. Once implanted in subjects, the devices may remain in place for indefinite durations with minimal risk of infection, since they can be refilled using a percutaneous needle. The devices have substantially anatomically-compatible shapes such that they are essentially non-detectable upon implantation in subjects, whereby they employ the skull space to avoid scalp or brain impingement. In addition to selectively administering therapeutic agents to subjects, the devices may also include an embedded imaging devices capable of providing image data to healthcare providers to monitor efficacy of treatment and/or need for repeat surgery. In some embodiments, the implants disclosed herein are used to replace missing skull segments, for example, from a previous surgical procedure, whereas in other exemplary embodiments, the implants are used intraoperatively following the removal of a skull bone flap.

In one aspect, this disclosure provides a magnetic resonance imaging (MRI) compatible, convection-enhanced delivery (CED) cranial implant device at least one cranial implant housing configured for intercranial implantation in at least one cranial opening of a subject. The cranial implant housing comprises a substantially anatomically-compatible shape, at least first and second surfaces, and at least one fluidic circuit comprising at least one cavity and at least one port that fluidly communicates with the cavity through at least the second surface in which the cavity comprises, or is capable of comprising, at least one fluidic therapeutic agent. The device also includes at least one CED pump operably connected to the fluidic circuit. The CED pump is configured to convey the fluidic therapeutic agent from the cavity through at least one fluid conduit when the fluid conduit is operably connected to the port to maintain at least one positive pressure gradient of the fluidic therapeutic agent at least proximal to an outlet of the fluid conduit. The device also includes at least one controller operably connected at least to the CED pump. The controller is configured to selectively effect the CED pump to convey the fluidic therapeutic agent through the fluid conduit when the fluid conduit is operably connected to the port and the cavity comprises the fluidic therapeutic agent. In addition, the device also includes at least one power source operably connected at least to the controller. The cranial implant housing, the CED pump, the controller, and the power source are fabricated from one or more MRI compatible materials. In certain embodiments, the cranial implant housing comprises a standardized form (i.e., off the shelf availability), whereas in other embodiments, the cranial implant housing comprises a form that is customized and patient-specific for the subject. Generally, the intercranial implantation is of an indefinite duration.

In some embodiments, the fluidic therapeutic agent comprises an optogenetic protein, a stem cell, an immune cell, an antibody, an enzyme, a radiation therapeutic agent, a chemical therapeutic agent, a neurological medicine, a neurological preventative medicine, a neurological enhancer, or combinations thereof. In certain embodiments, the fluidic therapeutic agent comprises one or more therapies selected from the group consisting of anti-tumor, anti-seizure, anti-Parkinson, anti-Huntington, anti-hydrocephalus, anti-ADHD, anti-Alzheimer's, anti-pain, anti-insomnia, anti-depression, anti-schizophrenia, energy-enhancing, mind-enhancing, neuro-protective, memory-enhancing, and combinations thereof.

The fluidic circuit typically comprises one or more fluidic channels operably connected to the cavity and port. The cranial implant housing optionally comprises multiple cavities that each comprise, or are capable of comprising, one or more fluidic therapeutic agents and/or other fluidic materials. In some embodiments, the cranial implant includes multiple ports that fluidly communicate with the cavity through at least the second surface. In certain embodiments, the cranial implant housing comprises an MRI compatible polymer, an MRI compatible metal, an MRI compatible bioengineered material, or combinations thereof. In some embodiments, the cranial implant housing comprises one or more of medical-grade titanium, titanium mesh, porous hydroxyapatite (HA), polymethylmethacrylate (PMMA), polyether ether ketone (PEEK), porous polyethylene, cubic zirconia (CZ), or combinations thereof. In some instances, the cranial implant housing comprises a substantially translucent material.

Typically, the cranial implant device includes at least one attachment mechanism or portion thereof operably connected, or connectable, to the cranial implant housing and/or the fluid conduit. The attachment mechanism or portion thereof is configured to attach the fluid conduit to the cranial implant housing such that the fluid conduit fluidly communicates with the fluidic circuit to enhance visible translucency and/or sonolucency.

The CED pump, the controller, and the power source are encased within the cranial implant housing. In some embodiments, for example, the CED pump, the controller, and the power source and optionally one or more other device components are encased within the cranial implant housing to maximize use of dead space between the first and second surfaces. In certain embodiments, the CED pump comprises at least one electroactive polymer (EAP) valve-gated pump. Typically, the controller is configured for wireless connectivity so as to be remotely monitored, activated, or both. In some embodiments, the power source comprises at least one battery (e.g., a zero-volt battery, a rechargeable battery, and/or the like). In certain embodiments, the cranial implant device includes at least one self-sealing access port disposed at least partially in or through the first surface. The self-sealing access port fluidly communicates with the cavity and is configured to receive one or more syringe needles (e.g., self-sealing syringe needles) through the scalp of the subject to add and/or remove the fluidic therapeutic agent to/from the cavity. In some of these embodiments, the self-sealing access port comprises a septum.

In certain embodiments, the cranial implant device includes one or more detectors at least partially disposed within cranial implant housing and operably connected at least to the controller. The detectors are configured to detect information from the subject and/or the device, which information is selected from the group consisting of: a volume of fluidic therapeutic agent disposed in the cavity, a volume of fluidic therapeutic agent conveyed through the fluidic circuit, a pressure of the fluidic therapeutic agent within the fluidic circuit and/or proximal thereto, a leakage of the fluidic therapeutic agent from the fluidic circuit, a status of the power source, a device component malfunction, visual images of brain or brain cavity via an implanted imaging device, and a detectable signal from the subject. Generally, the detectable signal from the subject is characteristic of at least one neurologically-related disease, condition or disorder. In certain embodiments, the detectable signal from the subject comprises image data.

In some embodiments, the fluid conduit is operably connected to the port (e.g., during device fabrication). In other embodiments, fluid conduits are operably connected to ports in the operating room just prior to implantation. In certain embodiments, the fluid conduit delivers the fluidic therapeutic agent to a diseased portion of brain parenchyma, a dead-space cavity following brain tumor resection, and/or a blood vessel, neuron or ventricle of a brain. In some embodiments, the fluid conduit comprises a polymer tubing. In certain embodiments, the fluid conduit comprises a catheter. Typically, the fluid conduit is at least partially disposed within a cannula that is operably connected to the cranial implant housing. In some embodiments, the second surface of the cranial implant housing comprises 2, 3, 4, or 5 ports that fluidly communicate with one or more fluidic circuits disposed within the cranial implant housing. In some of these embodiments, the cranial implant device includes 2, 3, 4, or 5 fluid conduits operably connected to the ports for which is diagnostic or therapeutic in value.

In certain embodiments, the cranial implant device includes at least one electrode operably connected, or connectable, to the cranial implant housing and/or the controller. The electrode is configured to selectively transmit one or more electrical signals to the subject (e.g., to effect flow alterations or low-level medicine quantities). In some embodiments, at least a portion of the electrode is disposed within the cranial implant housing. In certain embodiments, at least a portion of the electrode extends from the second surface of the cranial implant housing.

In some embodiments, the cranial implant device includes at least one imaging device operably connected, or connectable, to the cranial implant housing and/or the controller, which imaging device is configured to selectively capture image data from the subject. In certain embodiments, the imaging device comprises a camera, ultrasound, or related technology. In some embodiments, at least a portion of the imaging device is disposed within the cranial implant housing. In certain embodiments, at least a portion of the imaging device extends from the second surface of the cranial implant housing. Optionally, the imaging device comprises an ultrasound or non-invasive imaging device. In some embodiments, the imaging device comprises an optical coherence tomography (OCT) device. In some embodiments, the image data comprises low-definition image data, whereas in other embodiments, the image data comprises high-definition image data. In some embodiments, the ultrasound has duplex capabilities to additionally detect changes in blood flow.

In another aspect, the application discloses a magnetic resonance imaging (MRI) compatible, convection-enhanced delivery (CED) cranial implant device that includes at least one cranial implant housing configured for intercranial implantation in at least one cranial opening of a subject. The cranial implant housing comprises a substantially anatomically-compatible shape (e.g., to prevent visible deformity and optimal biocompatibility), at least first and second surfaces, and at least one fluidic circuit comprising at least one cavity and at least one port that fluidly communicates with the cavity through at least the second surface in which the cavity comprises, or is capable of comprising, at least one fluidic therapeutic agent. The cranial implant device also includes at least one CED pump operably connected to the fluidic circuit. The CED pump is configured to convey the fluidic therapeutic agent from the cavity through at least one fluid conduit when the fluid conduit is operably connected to the port to maintain at least one positive pressure gradient of the fluidic therapeutic agent at least proximal to an outlet of the fluid conduit. In addition, cranial implant device also includes at least one power source operably connected at least to the CED pump. Typically, the cranial implant housing, the CED pump, and the power source are fabricated from one or more MRI compatible materials (e.g., to prevent interference with tumor bed surveillance).

In another aspect, the application discloses a cranial implant device that includes at least one cranial implant housing configured for intercranial implantation in at least one cranial opening of a subject. Typically, the cranial implant housing comprises a substantially anatomically-compatible shape (e.g., either one as off-the-shelf or another patient-specific form). The cranial implant device also includes at least two functional components at least partially disposed within the cranial implant housing. A first functional component comprises a fluid-based physiological condition intervention system that comprises at least one convection-enhanced delivery (CED) pump (e.g., an electroactive polymer (EAP) valve-gated pump) configured to convey at least one fluidic therapeutic agent from the first functional component to the subject through at least one fluid conduit. A second functional component comprises a non-fluid-based physiological condition intervention system configured to transmit one or more therapeutic signals from the second functional component to the subject through at least one non-fluid conduit. The cranial implant device also includes at least one power source (e.g., a zero-volt battery, wirelessly rechargeable battery, or the like) at least partially disposed within the cranial implant housing, which power source is operably connected to the functional components. Typically, the cranial implant housing, the functional components, and/or the power source are fabricated from one or more magnetic resonance imaging (MRI) compatible materials. In some embodiments, for example, the cranial implant housing, the functional components, and/or the power source comprises an MRI compatible polymer, an MRI compatible metal, an MRI compatible bioengineered material, or combinations thereof. Optionally, the cranial implant housing, the functional components, and/or the power source comprises one or more of medical-grade titanium, titanium mesh, porous hydroxyapatite (HA), polymethylmethacrylate (PMMA), polyether ether ketone (PEEK), porous polyethylene, cubic zirconia (CZ), or combinations thereof.

In some embodiments, the cranial implant housing comprises at least first and second surfaces, and at least one fluidic circuit comprising at least one cavity and at least one port that fluidly communicates with the cavity through at least the second surface in which the cavity comprises, or is capable of comprising, the fluidic therapeutic agent. In certain embodiments, the CED pump is operably connected to the fluidic circuit. Typically, the fluidic circuit comprises one or more fluidic channels operably connected to the cavity and port. In some embodiments, the cranial implant housing includes at least one self-sealing access port disposed at least partially in or through the first surface, which self-sealing access port fluidly communicates with the cavity and is configured to receive one or more syringe needles (e.g., self-sealing syringe needles) through the scalp of the subject to add and/or remove the fluidic therapeutic agent to/from the cavity, and/or cell pathology from nearby catheter placement.

In certain embodiments, the functional components are configured to deliver one or more therapies to the subject selected from the group consisting of anti-tumor, anti-seizure, anti-Parkinson, anti-Huntington, anti-hydrocephalus, anti-ADHD, anti-Alzheimer's, anti-pain, anti-insomnia, anti-depression, anti-schizophrenia, energy-enhancing, mind-enhancing, neuro-protective, memory-enhancing, and combinations thereof. In some embodiments, the functional components and the power source are encased within the cranial implant housing.

In some embodiments, the cranial implant device includes at least one controller at least partially disposed within the cranial implant housing, which controller is operably connected to the functional components and the power source, and is configured to selectively effect the CED pump of first functional component to convey the fluidic therapeutic agent through the fluid conduit to the subject and the second functional component to transmit the therapeutic signals through the non-fluid conduit to the subject. The controller is typically configured for wireless connectivity so as to be remotely monitored, activated, adjusted, and/or charged. In some embodiments, for example, device infusion rated, dosage, and/or timing are changed via a wireless connection, typically depending upon certain treatment efficacy, patient symptoms, tumor growth, and/or vital signs. In certain of these embodiments, fluid conveyance involves remotely selecting a single or multiple catheters operably connected to a given implant device through which to pump fluid based, for example, on monitored flow and/or the like.

The first functional component generally comprises one or more detectors at least partially disposed within cranial implant housing and operably connected at least to the controller. The detectors are configured to detect information from the subject and/or the device, which information is selected from the group consisting of: a volume of fluidic therapeutic agent disposed in a cavity of the device, a volume of fluidic therapeutic agent conveyed through a fluidic circuit, a pressure of the fluidic therapeutic agent within the fluidic circuit and/or proximal thereto, a leakage of the fluidic therapeutic agent from the fluidic circuit, a status of the power source, a device component malfunction, and a detectable signal from the subject.

Typically, the fluid conduit and/or the non-fluid conduit extend from the cranial implant housing. In some embodiments, the fluid conduit and the non-fluid conduit are configured for fluidic, electrical, magnetic, imaging, and optical communication between the functional components and the subject. In some embodiments, the therapeutic signals comprise an electrical signal, a magnetic signal, an optical signal, an imaging signal, or combinations thereof. Optionally, the second functional component comprises at least one detector that is configured to detect information from the subject and/or the device. In some embodiments, the functional components are configured to provide acute neurological intervention comprising medicinal therapy, electro-stimulation therapy, radiation therapy, chemotherapy, radiation therapy, or a combination thereof. In certain embodiments, one or more of the functional components comprises a vital sign monitor, an optical coherence tomography (OCT) image monitor, a high definition camera, an intracranial pressure (ICP) monitor, an electroencephalography sensor (ECOG), some radiation seeds for local therapy, and/or a remote imaging monitor.

In some embodiments, the second functional component is configured to provide neuron modulation via optic sensors. Typically, the second functional component is configured for computerized monitoring of at least one physiological condition. In some embodiments, the second functional component is configured to monitor a diseased portion of brain parenchyma, a dead-space cavity following brain tumor resection, and/or a blood vessel (e.g., a feeding blood vessel), neuron or ventricle of a brain. Optionally, the second functional component comprises at least one intercranial pressure (ICP) monitor. In some embodiments, the second functional component comprises at least one vital sign or brain function monitor. In certain embodiments, the second functional component comprises at least one imaging device. In some embodiments, the imaging device comprises a camera. In certain embodiments, the imaging device comprises an optical coherence tomography (OCT) device. In some embodiments, the imaging device comprises an ultrasound device with or without duplex capabilities. Optionally, the second functional component comprises an electrical system, a remote imaging system, a radiation therapy system, a responsive neurostimulation system, and/or a neuromodulation system. In some embodiments, the second functional component comprises a medicine delivery device, an electrical signal delivery device, image capture device, radioactive seed device, energy storage device, and/or a computing device. In certain embodiments, the second functional component comprises an electrical energy source, an electrical energy detector, electromagnetic energy source, and/or an electromagnetic energy detector. Typically, the electrical energy source is configured to generate an electrical signal, the electromagnetic energy source is configured to generate an optical signal, and wherein the electromagnetic energy detector is configured to capture image data.

In another aspect, the application discloses a method of treating a neurologically-related disease, condition or disorder of a subject that includes surgically implanting at least one cranial implant device in at least one cranial opening of the subject. The cranial implant device comprises at least one cranial implant housing that comprises a substantially anatomically-compatible shape (either a standard (i.e., off-the-shelf) design or a customized (i.e., patient-specific) design), at least first and second surfaces, and at least one fluidic circuit comprising at least one cavity and at least one port that fluidly communicates with the cavity through at least the second surface, in which the cavity comprises at least one fluidic therapeutic agent, and in which at least fluid conduit extends from the second surface and fluidly communicates with the fluidic circuit. The cranial implant device also includes at least one convection-enhanced delivery (CED) pump operably connected to the fluidic circuit, which CED pump is configured to convey the fluidic therapeutic agent from the cavity through the fluid conduit to maintain at least one positive pressure gradient of the fluidic therapeutic agent at least proximal to an outlet of the fluid conduit within a cranial cavity of the subject. The cranial implant device also includes at least one controller operably connected at least to the CED pump, which controller is configured to selectively effect the CED pump to convey the fluidic therapeutic agent through the fluid conduit. The cranial implant device additionally includes at least one power source operably connected at least to the controller. The cranial implant housing, the CED pump, the controller, and the power source are fabricated from one or more magnetic resonance imaging (MRI) compatible materials (e.g., to prevent interference with subsequent imaging). The method also includes conveying an effective amount of the fluidic therapeutic agent from the cavity through the fluid conduit to maintain the positive pressure gradient of the fluidic therapeutic agent at least proximal to the outlet of the fluid conduit within the cranial cavity of the subject, thereby treating the neurologically-related disease, condition or disorder of the subject.

In certain embodiments, the neurologically-related disease, condition or disorder comprises one or more of cancer (e.g., brain cancer), epilepsy, Parkinson's disease, Huntington's disease, hydrocephalus, attention deficit-hyperactivity disorder (ADHD), pain, Alzheimer's disease, insomnia, depression, manic depression, and schizophrenia. Optionally, the fluidic therapeutic agent comprises an optogenetic protein, a stem cell, an immune cell, an antibody, an enzyme, a radiation therapeutic agent, a chemical therapeutic agent, a neurological enhancing medicine, a neurological preventative medicine, or combinations thereof. Typically, the method includes conveying the effective amount of the fluidic therapeutic agent to a diseased portion of brain parenchyma, a dead-space cavity following brain tumor resection, and/or a blood vessel (e.g., a feeding blood vessel), neuron or ventricle of the brain of the subject.

In some embodiments, at least one self-sealing access port is disposed at least partially in or through the first surface of the cranial implant housing, which self-sealing access port fluidly communicates with the cavity, and the method comprises inserting a syringe needle (e.g., a self-sealing syringe needle) through the scalp of the subject (e.g., above or around the device) and through the self-sealing access port, and adding the fluidic therapeutic agent to the cavity (e.g., an embedded cavity). In certain embodiments, the controller is configured for wireless connectivity so as to be remotely monitored, activated, and/or adjusted, and the method comprises wirelessly sending and/or receiving information and/or instructions to/from the controller.

In certain embodiments, the cranial implant device comprises one or more detectors at least partially disposed within cranial implant housing and operably connected at least to the controller, which detectors are configured to detect information from the subject and/or the device. In these embodiments, the method typically comprises detecting a volume of fluidic therapeutic agent disposed in the cavity, a volume of fluidic therapeutic agent conveyed through the fluidic circuit, a pressure of the fluidic therapeutic agent within the fluidic circuit and/or proximal thereto, a leakage of the fluidic therapeutic agent from the fluidic circuit, a status of the power source, a device component malfunction, and/or a detectable signal from the subject. In some embodiments, the cranial implant device comprises at least one intercranial pressure (ICP) monitor operably connected to the controller, and the method comprises monitoring the ICP of the subject using the ICP monitor (e.g., to detect pseudotumor cerebri, NPH, or obstructive hydrocephalus). In certain embodiments, the cranial implant device comprises at least one vital sign monitor operably connected to the controller, and the method comprises monitoring one or more vital signs of the subject using the vital sign monitor. In some embodiments, the cranial implant device comprises at least one imaging device operably connected to the controller, and the method comprises capturing image data from the subject using the imaging device. In some embodiments, the imaging device comprises an optical coherence tomography (OCT) device, and the method comprises capturing OCT image data from the subject using the OCT device. In certain embodiments, the imaging device comprises an ultrasound device with or without duplex capabilities, and the method comprises capturing ultrasound image data from the subject using the ultrasound device. In some embodiments, the method includes obtaining one or more MRI images of a cranial cavity of the subject.

In another aspect, the application discloses a method of monitoring therapeutic agent administration in a plurality of subjects that includes surgically implanting at least one cranial implant device in each of the plurality subjects (e.g., to assist with clinical research and/or controlled trials). Each of the cranial implant devices comprises at least one cranial implant housing that comprises a substantially anatomically-compatible shape (either a standard (i.e., off-the-shelf) design or a customized (i.e., patient-specific) design), at least first and second surfaces, and at least one fluidic circuit comprising at least one cavity and at least one port that fluidly communicates with the cavity through at least the second surface, wherein the cavity comprises at least one fluidic therapeutic agent, and wherein at least fluid conduit extends from the second surface and fluidly communicates with the fluidic circuit. The cranial implant device also includes at least one convection-enhanced delivery (CED) pump operably connected to the fluidic circuit. The CED pump is configured to convey the fluidic therapeutic agent from the cavity through the fluid conduit to maintain at least one positive pressure gradient of the fluidic therapeutic agent at least proximal to an outlet of the fluid conduit within a cranial cavity of a given subject. In certain embodiments, reversible pressure is used to create a vacuum for cytology retrieval or the like. The cranial implant device also includes at least one controller operably connected at least to the CED pump, which controller is configured to selectively effect the CED pump to convey the fluidic therapeutic agent through the fluid conduit, and for wireless connectivity so as to be remotely monitored, activated, and/or adjusted. The cranial implant device also includes at least one power source operably connected at least to the controller. The cranial implant housing, the CED pump, the controller, and the power source are fabricated from one or more magnetic resonance imaging (MRI) compatible materials. The method also includes conveying selected amounts of the fluidic therapeutic agent to one or more members of the plurality of subjects using the implanted cranial implant devices. In addition, the method also includes gathering data from one or more selected sets of members of the plurality of subjects using the wireless connectivity of the implanted cranial implant devices, thereby monitoring the therapeutic agent administration in the plurality of subjects (e.g., by way of a clinical trial investigation). In some embodiments, the data correlates with a measure of efficacy and/or toxicity of the therapeutic agent in the plurality of subjects. In certain embodiments, the data correlates with a measure of performance of the cranial implant devices in the plurality of subjects.

In another aspect, the application discloses a surgical method that includes surgically implanting at least one cranial implant device in at least one cranial opening of the subject. The cranial implant device comprises at least one cranial implant housing that comprises a substantially anatomically-compatible shape (either a standard (i.e., off-the-shelf) design or a customized (i.e., patient-specific) design), at least first and second surfaces, and at least one fluidic circuit comprising at least one cavity and at least one port that fluidly communicates with the cavity through at least the second surface in which the cavity comprises at least one fluidic therapeutic agent, and in which at least fluid conduit extends from the second surface and fluidly communicates with the fluidic circuit. The cranial implant device also includes at least one CED pump operably connected to the fluidic circuit. The CED pump is configured to convey the fluidic therapeutic agent from the cavity through the fluid conduit to maintain at least one positive pressure gradient of the fluidic therapeutic agent at least proximal to an outlet of the fluid conduit within a cranial cavity of the subject, and may also be used to maintain transient negative pressure for cell cytometry retrieval in some embodiments. The cranial implant device also includes at least one controller operably connected at least to the CED pump. The controller is configured to selectively effect the CED pump to convey the fluidic therapeutic agent through the fluid conduit. The cranial implant device also includes at least one power source operably connected at least to the controller. The cranial implant housing, the CED pump, the controller, and the power source are fabricated from one or more magnetic resonance imaging (MRI) compatible materials (e.g., to prevent inference with related imaging processes).

In another aspect, the application discloses a method of fabricating a cranial implant device that includes forming at least first and second portions of a cranial implant housing, wherein once assembled, the first and second portions form at least one cavity and at least one port that fluidly communicates with the cavity through at least one surface of the cranial implant housing to thereby generate at least one fluidic circuit, and wherein the first and second portions are formed from one or more magnetic resonance imaging (MRI) compatible materials. The method also includes positioning at least one convection-enhanced delivery (CED) pump relative to the first and/or second portions, wherein the CED pump is formed from one or more MRI compatible materials, positioning at least one controller relative to the first and/or second portions and operably connecting the controller to the CED pump, wherein the controller is formed from one or more MRI compatible materials, and positioning at least one power source relative to the first and/or second portions and operably connecting the power source to the controller, wherein the power source is formed from one or more MRI compatible materials. In addition, the method also includes attaching the first and second portions of a cranial implant housing to one another to generate the fluidic circuit and such that the CED pump, the controller, and the power source are encased within the first and second portions, and such that the cranial implant housing comprises a substantially anatomically-compatible shape, thereby fabricating the cranial implant device (e.g., which mirrors or corresponds to the natural curvature and thickness of the human skull).

In another aspect, the application discloses an electroactive polymer (EAP) valve-gated pump that includes a top housing structure comprising at least a top surface in which at least one top orifice is disposed through the top surface. The pump also includes a bottom housing structure comprising a substantially concave fluid chamber having a top opening in which at least first and second fluid channels fluidly communicate with the fluid chamber. In addition, the pump also includes a membrane portion disposed between the top and bottom housing structures, which membrane portion encloses the concave fluid chamber when the top and bottom housing structures are attached to one another. The pump also includes an EAP-actuation mechanism (e.g., a dielectric EAP-actuation mechanism, an ionic EAP-actuation mechanism, etc.) operably connected to the membrane portion. The EAP-actuation mechanism is configured to displace the membrane portion to thereby effect fluid conveyance (e.g., in either direction through the pump). In some embodiments, the top and bottom housing structures comprise one or more reversible attachment features configured to reversibly attach the top and bottom housing structures to one another. In certain embodiments, the membrane portion comprises a silicon or other resealable membrane. In other exemplary embodiments, a cranial implant device comprises the pump. In these embodiments, at least a first fluid conduit is operably connected to the first fluid channel of the bottom housing structure and to a cavity disposed within the cranial implant device. In these embodiments, at least a second fluid conduit is also operably connected to the second fluid channel of the bottom housing structure and extends from a port disposed through at least one surface of the cranial implant device. In these embodiments, the pump is also operably connected to a controller disposed within the cranial implant device.

In another aspect, the disclosure provides a convection-enhanced delivery (CED) cranial implant device that includes at least one cranial implant housing configured for intercranial implantation in at least one cranial opening of a subject (e.g., which typically matches the thickness of the removed or missing skull segment). The cranial implant housing comprises a substantially anatomically-compatible shape (either a standard (i.e., off-the-shelf) design or a customized (i.e., patient-specific) design), at least first and second surfaces, and at least one fluidic circuit comprising at least one cavity and at least one port that fluidly communicates with the cavity through at least the second surface, wherein the cavity comprises, or is capable of comprising, at least one fluidic therapeutic agent. The CED cranial implant device also includes at least one CED pump operably connected to the fluidic circuit, which CED pump is configured to convey the fluidic therapeutic agent from the cavity through at least one fluid conduit when the fluid conduit is operably connected to the port to maintain at least one positive pressure gradient of the fluidic therapeutic agent at least proximal to an outlet of the fluid conduit. In some embodiments, the device is configured to selectively effect reverse pressure application for transient suction for cell retrieval. The CED cranial implant device also includes at least one controller operably connected at least to the CED pump, which controller is configured to selectively effect the CED pump to convey the fluidic therapeutic agent through the fluid conduit when the fluid conduit is operably connected to the port and the cavity comprises the fluidic therapeutic agent, and at least one power source operably connected at least to the controller. In addition, one or more of the cranial implant housing, the CED pump, the controller, the power source, or sub-components thereof, are fabricated from one or more non-MRI compatible materials, which non-MRI compatible materials are selectively and reversibly removable from the CED cranial implant device when the CED cranial implant device is implanted in the subject.

In another aspect, the disclosure provides a convection-enhanced delivery (CED) implant device that includes at least one implant housing configured for implantation in at least one opening (e.g., a thoracic opening, an abdominal opening, etc.) of a subject. The implant housing comprises a substantially anatomically-compatible shape, at least first and second surfaces, and at least one fluidic circuit comprising at least one cavity and at least one port that fluidly communicates with the cavity through at least the second surface, wherein the cavity comprises, or is capable of comprising, at least one fluidic therapeutic agent. The CED implant device also includes at least one CED pump operably connected to the fluidic circuit, which CED pump is configured to convey the fluidic therapeutic agent from the cavity through at least one fluid conduit when the fluid conduit is operably connected to the port to maintain at least one positive pressure gradient of the fluidic therapeutic agent at least proximal to an outlet of the fluid conduit. The CED implant device also includes at least one controller operably connected at least to the CED pump, which controller is configured to selectively effect the CED pump to convey the fluidic therapeutic agent through the fluid conduit when the fluid conduit is operably connected to the port and the cavity comprises the fluidic therapeutic agent, and at least one power source (e.g., a wirelessly rechargeable battery or the like) operably connected at least to the controller. In addition, the implant housing, the CED pump, the controller, and the power source are fabricated from one or more MRI compatible materials.

### BRIEF DESCRIPTION OF THE DRAWINGS

The accompanying drawings, which are incorporated in and constitute a part of this specification, illustrate certain embodiments, and together with the written description, serve to explain certain principles of the cranial implant devices, pumps, and related methods disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 schematically shows a method of attaching a left-sided, posterior, full-thickness skull resection outlined by a cut region and a CED cranial implant device being inserted into the resected portion of the removed or missing skull according to one exemplary embodiment.

FIG. 2 schematically shows the resulting intercranial implantation of the CED cranial implant device of FIG. 1 with rigid fixation.

FIG. 3A schematically depicts a CED cranial implant device from a perspective view according to one exemplary embodiment.

FIG. 3B schematically shows the CED cranial implant device of FIG. 3A from an exploded perspective view.

FIG. 3C schematically shows the CED cranial implant device of FIG. 3A from an exploded top view.

FIG. 3D schematically shows the CED cranial implant device of FIG. 3A from an exploded top view.

FIG. 3E schematically shows the CED cranial implant device of FIG. 3A from a top view.

FIG. 4A schematically illustrates a component cavity from the cranial implant housing of the CED cranial implant device of FIG. 3A from a bottom view.

FIG. 4B schematically illustrates the component cavity of FIG. 4A from a top view.

FIG. 4C schematically shows the component cavity of FIG. 4A from a side view.

FIG. 4D schematically illustrates the component cavity of FIG. 4A from a perspective view.

FIG. 5A schematically illustrates a fluidic therapeutic agent cavity from the cranial implant housing of the CED cranial implant device of FIG. 3A from a perspective view.

FIG. 5B schematically shows the fluidic therapeutic agent cavity of FIG. 5A from a side view.

FIG. 5C schematically depicts the fluidic therapeutic agent cavity of FIG. 5A from a top view.

FIG. 6A schematically shows an electroactive polymer (EAP) valve-gated pump from a side view according to one exemplary embodiment.

FIG. 6B schematically illustrates top and bottom housing structures from the pump of FIG. 6A from an exploded perspective view.

FIG. 6C schematically illustrates the top housing structure from FIG. 6B from a top view.

FIG. 6D schematically illustrates the top housing structure from FIG. 6B from a side view.

FIG. 6E schematically illustrates the bottom housing structure from FIG. 6B from a top view.

FIG. 6F schematically illustrates the top housing structure from FIG. 6B from a side view.

FIG. 6G schematically shows a dielectric EAP-actuation mechanism from a top view according to one exemplary embodiment.

FIG. 6H schematically shows an electroactive polymer (EAP) valve-gated pump from a bottom view according to one exemplary embodiment.

FIG. 6I schematically shows the EAP valve-gated pump of FIG. 6H from a bottom view.

FIG. 6J schematically shows the EAP valve-gated pump of FIG. 6H from a side view.

FIG. 7 schematically shows a wireless communication network for gathering data from one or more subjects having intercranially implanted CED cranial implant devices according to one exemplary embodiment.

FIG. 8 schematically shows a CED cranial implant device being inserted into a resected portion of a removed or missing skull according to one exemplary embodiment.

FIGS. 9A-C schematically show an electroactive polymer (EAP) configuration according to one exemplary embodiment. FIG. 9A schematically shows the EAP from a sectional view. FIG. 9B schematically shows a detailed view of the EAP from FIG. 9A in the absence of an applied current. FIG. 9C schematically shows a detailed view of the EAP from FIG. 9A under the application of a current, which causes ions to shift and induce the polymer to bend, thereby effecting a pressure difference that induces fluid conveyance in the implant devices described herein.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, cranial implant devices, and component parts, the following terminology, and grammatical variants thereof, will be used in accordance with the definitions set forth below.

About: As used herein, “about” or “approximately” as applied to one or more values or elements of interest, refers to a value or element that is similar to a stated reference value or element. In certain embodiments, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 4%, 3%, 2%, 1%, or less in either direction (greater than or less than) of the stated reference value or element unless otherwise stated or otherwise evident from the context (except where such number would exceed 100% of a possible value or element).

Administer: As used herein, “administering” a composition or therapeutic agent to a subject means to give, apply or bring the composition into contact with the subject. Administration can be accomplished by any of a number of routes, including, for example, topical, oral, subcutaneous, intercranial, intramuscular, intraperitoneal, intravenous, intrathecal and intradermal.

Customized: As used herein, “customized” in the context of cranial implant shapes refers to a shape that has been created at the point of fabrication specifically for an individual subject. In some embodiments, for example, custom craniofacial implants (CCIs) are designed and manufactured using computer-aided design/manufacturing (CAD/CAM) based in part on fine cut preoperative computed tomography (CT) scans and three-dimensional reconstruction (+/−stereolithographic models).

Detect: As used herein, “detect,” “detecting,” or “detection” refers to an act of determining the existence or presence of one or more characteristics, properties, states, or conditions in a subject, in a sample obtained or derived from a subject, or in a device, system, or component thereof.

Functional Component: As used herein, “functional component” means any therapeutic hardware or compositions including, but not limited to, medicines to treat any patient-specific illness, or electronic, mechanical, imaging modality and/or electro-mechanical device to remotely monitor (e.g., via Wi-Fi connectivity) or intervene any specific neurologic illness, including imaging, monitoring, electrostimulation, radiation therapy, polarized light/laser neuronal modulation devices.

Standardized: As used herein, “standardized” in the context of cranial implant shapes refers to a shape that has not been created at the point of fabrication specifically for any individual subject. Instead, a standardized implant shape is typically selected for ease of readily reproducible manufacture. Cranial implants having standardized shapes may also be referred to as “off the shelf” neurological implants.

Subject: As used herein, “subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.” For example, a subject can be an individual who has been diagnosed with having a cancer, is going to receive a cancer therapy, and/or has received at least one cancer therapy. The subject can be in remission of a cancer.

Substantially Anatomically-Compatible Shape: As used herein, “substantially anatomically-compatible shape” in the context of cranial implant devices refers to a shape such that when the device is implanted in a subject, the device is essentially visually imperceptible in the absence of, for example, analytical imaging, such as X-ray-based imaging or the like.

### DETAILED DESCRIPTION

This application discloses magnetic resonance imaging (MRI) compatible, convection-enhanced delivery (CED) cranial implant devices and related methods for performing a wide array of therapeutic and/or monitoring applications. Once implanted in subjects, the devices may remain in place for indefinite durations. The devices have substantially anatomically-compatible shapes such that they are essentially visually non-detectable to the naked eye upon implantation in subjects and safely avoid pressure on the scalp above or brain below. In addition to selectively administering therapeutic agents to subjects, the devices also typically include imaging devices that provide image data to patients, patient family/friends, healthcare providers to monitor courses of treatment. The implantable devices described herein typically include low-profiles (e.g., to avoid scalp-related complications and high extrusion risk leading to premature explantation). Optionally, the devices described herein are configured for implantation elsewhere in a patient's body, such as in the thoracic cavity (e.g., to treat cardiovascular or pulmonary disease), the abdominal cavity (e.g., to treat hepatological disease), or the pelvic cavity (e.g., to treat ovarian, uterine or prostatic disease) for non-brain related pathologies and chronic illnesses. In these embodiments, the implant devices are also typically configured to be MRI compatible.

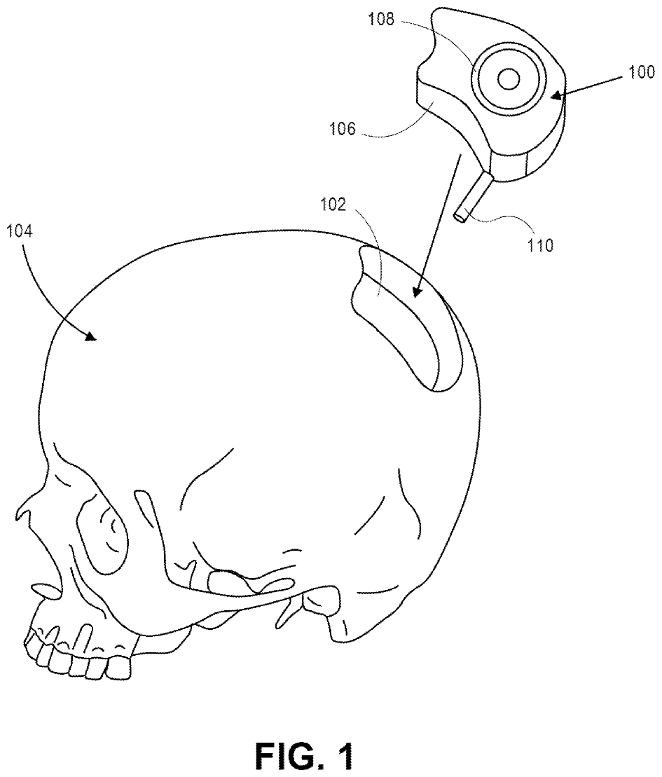

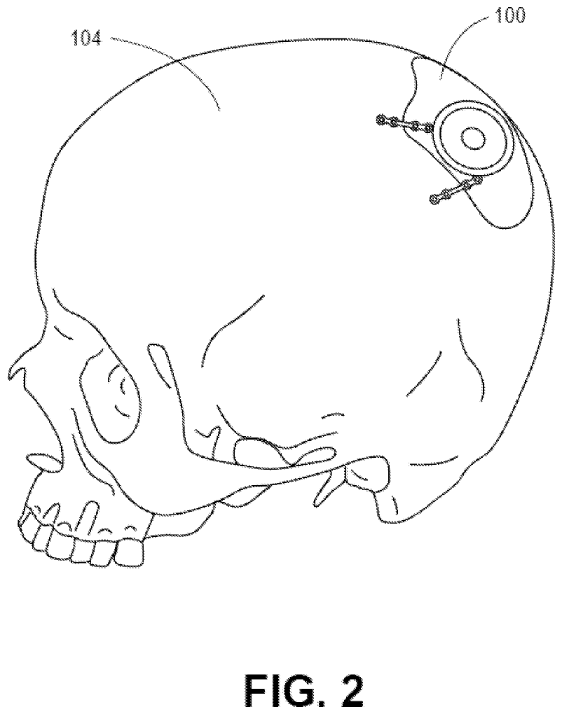

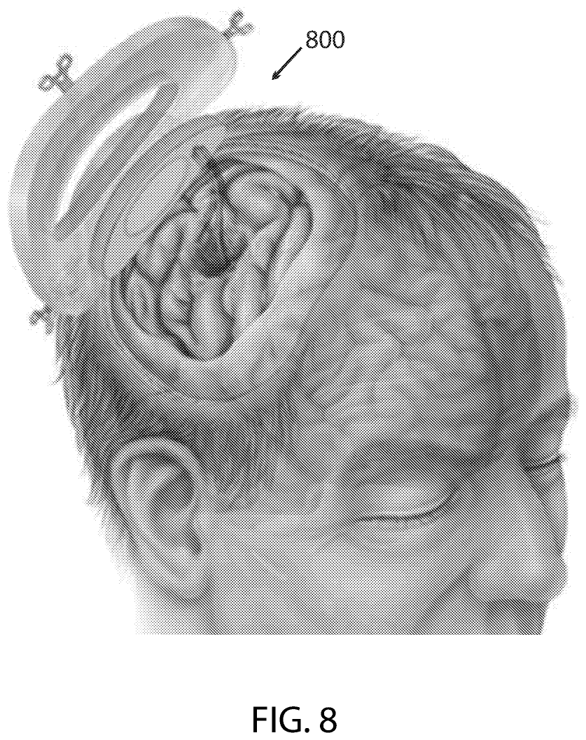

By way of overview, FIGS. 1 and 2 schematically show the insertion of cranial implant device 100 (e.g., fabricated from MRI-compatible materials) in resected or missing portion 102 of skull 104 during a surgical procedure, such as a surgical implantation procedure for various forms of neuroplastic surgery, craniomaxillofacial surgery and/or neurosurgery including an implant-based cranioplasty according to one exemplary embodiment. To further illustrate, FIG. 8 also schematically shows CED cranial implant device 800 being inserted into a resected portion of a removed or missing skull according to one exemplary embodiment. In certain exemplary embodiments, the cranial implant devices described herein are miniaturized and implanted within a patient's own bone flap being replaced following a common neurosurgical craniotomy. In some of these embodiments, a given cranial implant device may be partly or fully recessed within the undersurface of one's own bone flap following craniotomy and replaced accordingly. As shown, cranial implant device 100 includes cranial implant housing 106, which includes a form or shape that is customized for missing or resected portion 102 of skull 104. In some of these embodiments, for example, a given cranial implant device may be embedded within the skull space as either a universal or standard design or a patient-specific implant device using a customized design following computer-assisted design and modeling for patient-specific dimensions. In other embodiments, cranial implant housings are fabricated with standardized forms (e.g., an off the shelf modular design that may be universal or standard and embedded within a skull space as a stand-alone device), the shapes of which are optionally further modified prior to surgical implantation. As also shown, cranial implant device 100 also includes functional component 108, which fluidly communicates with fluid conduit 110. Fluid conduit 110 is typically of a selected length and disposed at an angle relative to cranial implant housing 106 such that an outlet of fluid conduit 110 is positioned at a desired location within the cranial cavity of skull 104 (e.g., a diseased portion of brain parenchyma, a dead-space cavity following brain tumor resection, and/or a blood vessel (e.g., a feeding blood vessel), neuron or ventricle of the brain). As described further herein, functional component 108 typically includes a fluid-based physiological condition intervention system that includes a convection-enhanced delivery (CED) pump configured to convey one or more fluidic therapeutic and/or diagnostic agents (e.g., an optogenetic protein, a stem cell, an immune cell, an antibody, an enzyme, a saline solution, a vitamin, a supplement, a dye (e.g., an acoustically activated dye or the like), a radiation therapeutic agent, a chemical therapeutic agent, a neurological medicine, a neurological preventative medicine, or combinations thereof) through fluid conduit 110 once cranial implant device 100 is implanted. To further illustrated, various therapies are optionally administered to subjects using the cranial implant devices disclosed herein, including, for example, anti-tumor, anti-seizure, anti-Parkinson, anti-hydrocephalus, anti-ADHD, anti-Alzheimer's, anti-pain, anti-insomnia, anti-depression, anti-schizophrenia, energy-enhancing, mind-enhancing, memory-enhancing, neuro-protective, anti-Huntington's, anti-aging, and/or like.

In certain embodiments, other or additional functional components are included in the cranial implant devices disclosed in this application, such as various non-fluid-based physiological condition intervention systems. Typically, the intercranial implantation of the cranial implant devices described herein is intended to be for an indefinite duration to permit therapeutic administration for as long as needed. This feature overcomes significant limitations of many pre-existing CED applications, which can only typically remain in place for at most 5-10 days due to the risk of infection over longer periods of time and/or do not have enough positive pressure to overcome flow resistance with the human brain.

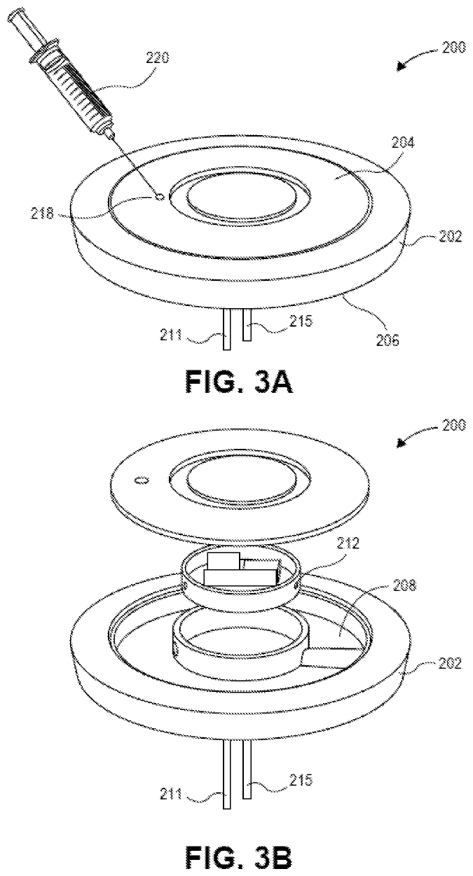

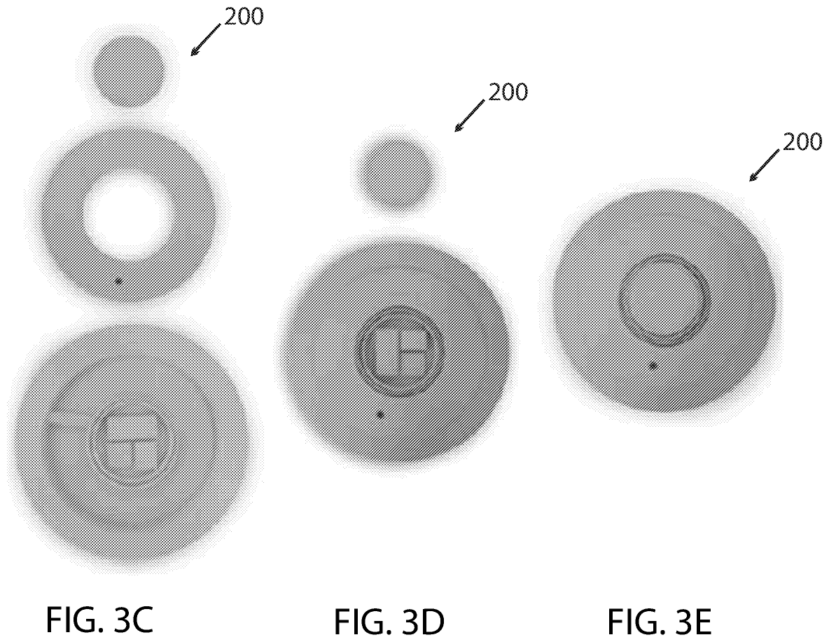

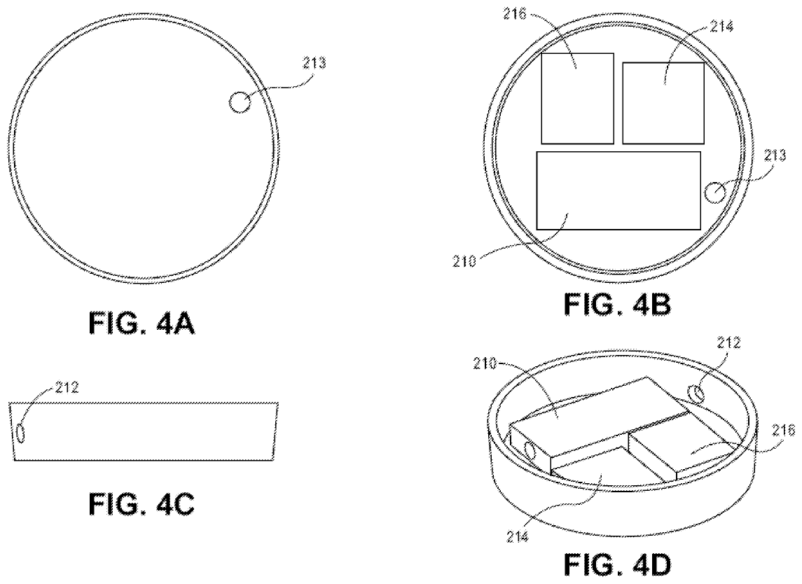

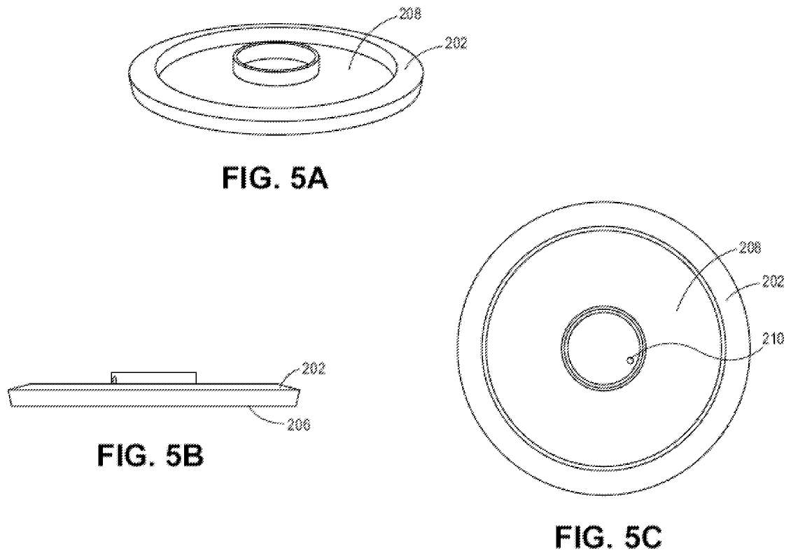

To further illustrate, FIGS. 3-5 schematically depict additional aspects of the MRI compatible CED cranial implant devices disclosed herein. As shown, CED cranial implant device 200 includes cranial implant housing 202, which in this exemplary embodiment has a standardized form, for example, for ease of manufacture. In this embodiment, cranial implant housing 202 is schematically shown as having a generally circular form (e.g., with a curvature matching the human skull). Essentially any standardized form is optionally utilized (e.g., elliptical, square, rectangular, triangular, and the like).

As shown, cranial implant housing 202 is configured for intercranial implantation in a cranial opening of a subject. Typically, cranial implant housing 202 has a substantially anatomically-compatible shape in order to be essentially imperceptible to the naked eye upon implantation in a subject with no visible deformity. Cranial implant housing 202 includes first and second surfaces, 204 and 206, respectively. Cranial implant housing 202 also includes a fluidic circuit that includes cavity 208 and port 213 that fluidly communicates with cavity 208 through second surface 206. Optionally, cavity 208 and port 213 fluidly communicate with one another through other surfaces of cranial implant housing 202. Cavity 208 is configured to contain fluidic therapeutic agents (e.g., chemotherapeutic agents, immunological agents, etc.) that are pre-loaded in cranial implant device 200 prior to implantation and/or added post-implantation in a subject. In some embodiments, fluidic circuits include one or more fluidic channels operably connected to cavities and ports, including, for example, microfluidic channel networks. In certain other exemplary embodiments, cranial implant housings include multiple cavities that each comprise, or are capable of comprising, one or more fluidic therapeutic agents and/or other fluidic materials. In some embodiments, cranial implant devices include multiple ports that fluidly communicate with cavities through, for example, second surface 206.

First surface 204 typically also includes self-sealing access port 218 (e.g., a septum or the like) disposed at least partially in or through first surface 204. Self-sealing access port 218 fluidly communicates with cavity 208 and is configured to repeatedly receive syringe needle 220 (e.g., a self-sealing syringe) through the scalp of the subject to add and/or remove fluidic therapeutic agents to/from cavity 208. Suitable self-sealing access ports are commercially available from various suppliers, including, for example, Smiths Medical. In certain embodiments, self-sealing access ports have contoured shapes for tactile recognition following device implantation. In other embodiments, a protective barrier (e.g., a titanium plate or the like) is position below self-sealing access port 218 in cavity 208 to prevent syringe needle 220 from damaging CED cranial implant device 200 upon insertion.

Cranial implant device 200 also includes CED pump 210 operably connected to the fluidic circuit and disposed within the implant core in this exemplary embodiment. Essentially any type of pump configuration is optionally adapted for use in the cranial implant devices disclosed herein, including gear pumps, vane pumps, hose pumps, centrifugal pumps, lobe pumps, diaphragm pumps, peristaltic pumps, positive displacement pumps, non-positive displacement pumps, and the like. In addition, a variety of actuators are optionally adapted to effect fluid conveyance using these pumps, including, for example, piezo electric motors, reciprocating motors, rotary motors, and the like. CED pump 210 is configured to convey the fluidic therapeutic agent from cavity 208 through a fluid conduit (not shown) that fluidly communicates with CED pump 210 and port 212, and fluid conduit 211 that fluidly communicates with CED pump 210 through port 213 disposed through second surface 206. In some embodiments, for example, fluid conduit 211 (e.g., a catheter or other polymer tubing) is operably connected to CED pump 210 and extends from cranial implant device 200 through port 213. In some embodiments, fluid conduit 211 is operably connected directly to port 213. CED pump 210 is configured to maintain positive pressure gradient of the fluidic therapeutic agent at least proximal to an outlet of the fluid conduit (e.g., to effect convection-enhanced delivery of the fluidic therapeutic agent). To provide a measure of rigidity for implantation, fluid conduits are typically at least partially disposed within cannulas that are operably connected to cranial implant housings. In other exemplary embodiments, second surface 206 includes 2, 3, 4, 5, or more ports that fluidly communicate with the fluidic circuits disposed within cranial implant housings. In these embodiments, the fluid conduits are typically operably connected to the ports and/or to CED pump 210, for example, via a manifold or the like. In some embodiments, pumps are configured to deliver fluid with positive pressure, pulsatile flow into the brain parenchyma, the lateral ventricle, a potential space following resection, a feeding blood vessel, and/or an artificial cavity, such as a refillable bladder. In certain embodiments, pumps are configured to selectively remove, aspirate (e.g., with negative pressure), or syphon extraneous fluid in a reversible manner from brain parenchyma, lateral ventricle, a potential space, brain tumor cavity, and/or an artificial cavity (e.g., a refillable bladder). In some of these embodiments, fluid is syphoned and removed by percutaneous needle puncture for sampling (e.g., cell sampling) and/or pumped back to the site of origin once the fluid is, for example, reconditioned or the like. In some embodiments, pumps are synergistically paired with one or more remote imaging devices to monitor fluid distribution using, for example, wireless connectivity.

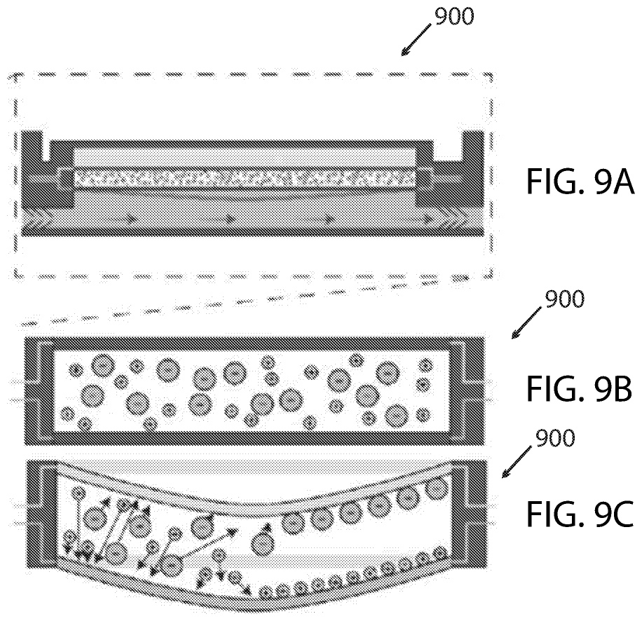

The present disclosure also provides electroactive polymer (EAP) valve-gated pumps (e.g., having dielectric or ionic EAP actuation mechanisms) that are optionally used in the CED cranial implant devices described herein or in essentially any other application to effect fluid conveyance (e.g., in other types of implantation devices to deliver therapeutic agents to parts of a patient's body, other than the brain). To further illustrate, FIGS. 9A-C schematically show an EAP configuration according to one exemplary embodiment. EAP powered valve-gated pumps, or other types of pumps, motors, or other related components, are typically fabricated from MRI-compatible materials (e.g., a transparent photopolymer or other material described herein or otherwise known to persons of ordinary skill in the art). The pumps are optionally configured to convey fluids at a variety of flow rates controlled, adjusted, and/or monitored remotely. In some embodiments where EAP valve-gated pumps are included in CED cranial implant devices, for example, relatively low flow rates of less than about 5 μL/minute (e.g., about 4 μL/minute, about 3 μL/minute, about 2 μL/minute, about 1 μL/minute) are used to maintain a constant pressure gradient at the site of a therapeutic application.

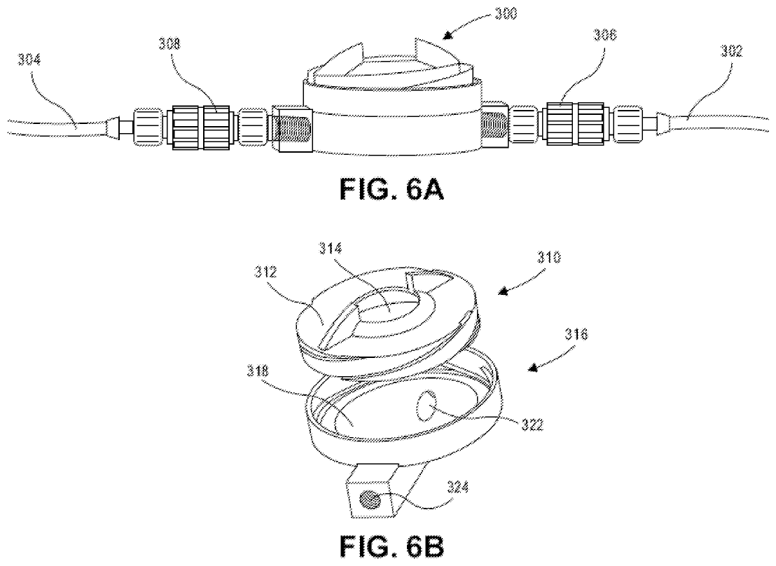

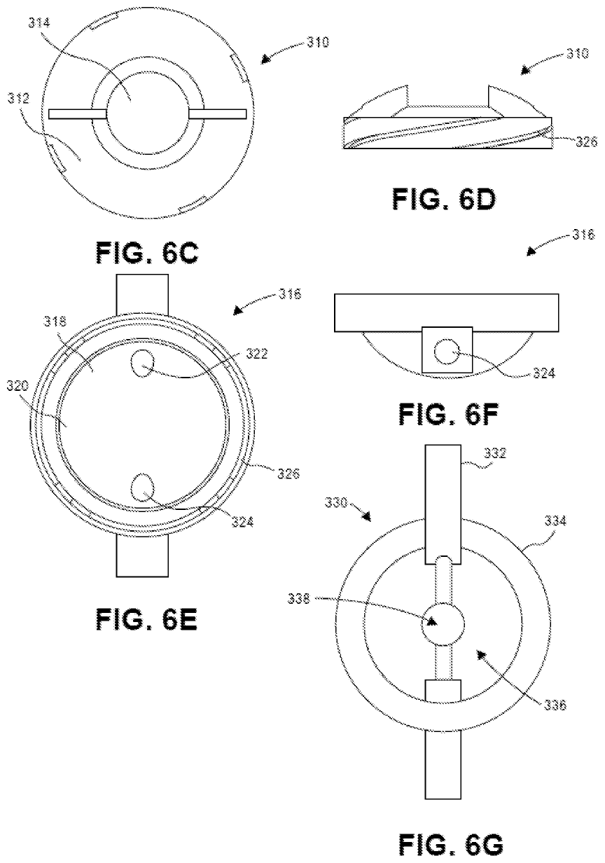

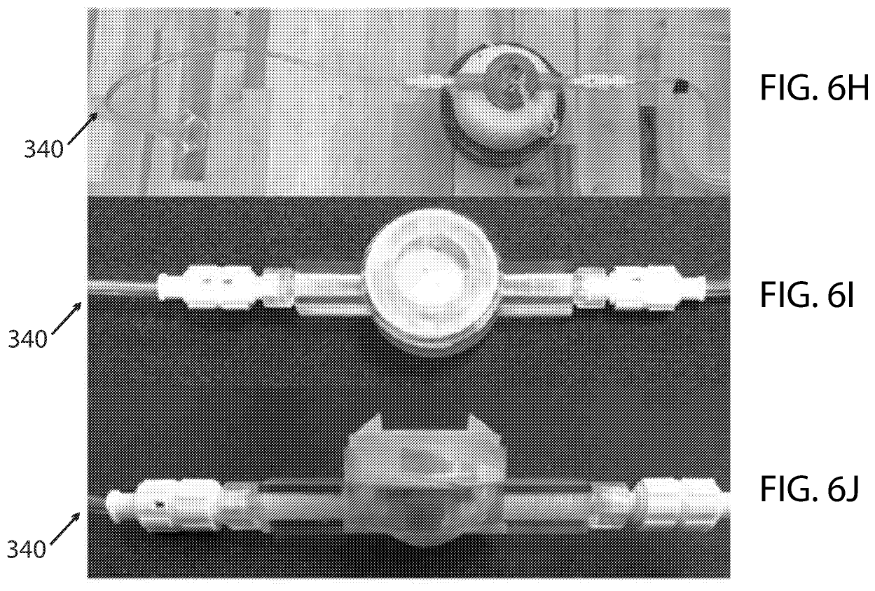

To further illustrate, FIG. 6 schematically shows exemplary EAP valve-gated pump 300. In FIG. 6A, for example, EAP valve-gated pump 300 is operably connected to catheters 302 and 304 via attachment mechanisms 306 and 308, respectively (shown as luer lock-type connections). As also shown, EAP valve-gated pump 300 includes top housing structure 310, which includes top surface 312 having top orifice 314 disposed through top surface 312. EAP valve-gated pump 300 also includes bottom housing structure 316, which includes substantially concave fluid chamber 318 having top opening 320. As also shown, first and second fluid channels 322 and 324, respectively, fluidly communicate with fluid chamber 318. Although not within view, EAP valve-gated pump 300 also includes a membrane portion (e.g., a silicon membrane or the like) disposed between the top and bottom housing structures. The membrane portion encloses concave fluid chamber 318 when the top and bottom housing structures 310 and 316, respectively, are attached to one another.

In certain embodiments, dielectric EAP-activation mechanisms are used with EAP valve-gated pump 300 to effect fluid conveyance. Optionally, ionic EAP-activation mechanisms are adapted for use. FIG. 6G schematically shows dielectric EAP-actuation mechanism 330. As shown, dielectric EAP-actuation mechanism 330 includes copper tap 332, acrylic frame 334, VHB membrane 336, and copper or carbon grease 338. EAP-actuation mechanism 330 is typically attached to the membrane portion mentioned above (e.g., a silicon membrane or the like). When the EAP-actuation mechanism 330 is actuated and contracts, the silicon membrane is displaced, thereby causing fluid to flow through pump 300. Electrical connections to EAP valve-gated pump 300 are not shown in the figures, but are well-known to persons having ordinary skill in the art. In some embodiments, for example, electrical wiring is disposed through top orifice 314 of EAP valve-gated pump 300. To further illustrate, FIGS. 6H-J schematically show electroactive polymer (EAP) valve-gated pump 340.

In some embodiments, top and bottom housing structures 310 and 316, respectively, include reversible attachment features 326 (shown as corresponding threaded regions) configured to reversibly attach top and bottom housing structures 310 and 316 to one another.

Cranial implant devices typically also include attachment mechanisms or portions thereof (e.g., a luer lock-type connections or the like) operably connected, or connectable, to the cranial implant housing and/or fluid conduits. These attachment mechanisms are generally configured to attach fluid conduits to the cranial implant devices such that the fluids conduit fluidly communicate with the fluidic circuits and to minimize the risk of joints becoming disconnected after placement.

In addition, cranial implant device 200 also includes controller 214 (e.g., a microcontroller or the like) operably connected at least to CED pump 210. Controller 214 is configured to selectively effect CED pump 210 to convey the fluidic therapeutic agent (e.g., at selected dosages and at defined times) through the fluid conduit through port 213 from cavity 208. Typically, controller 214 is configured for wireless connectivity so as to be remotely monitored, activated, and/or adjusted.

Cranial implant device 200 also includes power source 216 operably connected controller 214 and CED pump 210. Essentially any suitable power source (e.g., a rechargeable power source) is optionally used, or adapted, for use to provide power to the components of cranial implant device 200. In some exemplary embodiments, one or more batteries (e.g., zero-volt batteries, implantable batteries, rechargeable batteries, and/or the like) are used. Typically, power sources are rechargeable (e.g., a battery that is rechargeable via inductive or wireless charging) and safe wireless reactivation.

Cranial implant housing 202, CED pump 210, controller 214, and power source 216 of cranial implant device 200 are typically fabricated from one or more MRI compatible materials, for example, to permit on-going MRI monitoring of a given course of treatment for a subject while cranial implant device 200 remains implanted in the subject. Essentially any MRI compatible material is optionally used, or adapted for use, in manufacturing the cranial implant housings disclosed herein. In some embodiments, for example, the cranial implant housing comprises an MRI compatible polymer, an MRI compatible metal, an MRI compatible bioengineered material, or combinations thereof. To further illustrate, the cranial implant housing optionally includes medical-grade titanium, titanium mesh, porous hydroxyapatite (HA), polymethylmethacrylate (PMMA), polyether ether ketone (PEEK), porous polyethylene, cubic zirconia (CZ), or combinations thereof. In certain embodiments, cranial implant housings are fabricated from substantially translucent materials, for example, to facilitate visualization (e.g., via visual translucency and/or sonolucency) by the surgeon through the housing during and after implantation. Moreover, CED pumps, controllers, power sources, and other functional components are typically encased within cranial implant housings, for example, to prevent bodily fluids from contacting those components and/or to maximize use of dead space between the first and second surfaces of the housings. In certain embodiments, at least some implant device components are fabricated from non-MRI compatible materials. In these embodiments, those device components are typically selectively removable from the remainder of an implanted device to facilitate MRI processes. Device components (e.g., implant housings, pump components, and the like) are optionally formed by various fabrication techniques or combinations of such techniques including, e.g., 3D printing, cast molding, machining, stamping, engraving, injection molding, etching, embossing, extrusion, or other techniques well-known to persons of ordinary skill in the art.

In some embodiments, cranial implant device 200 includes other functional components, such as non-fluid-based physiological condition intervention systems configured to transmit therapeutic signals from the functional component to the subject and/or a remote receiver through a non-fluid conduit. In certain embodiments, for example, cranial implant device 200 includes one or more detectors or sensors at least partially disposed within cranial implant housing 202 and operably connected at least to controller 214. These detectors or sensors are typically configured to detect detectable signals or other information from the subject and/or the device. To illustrate, this information typically includes, for example, a volume of fluidic therapeutic agent disposed in cavity 208, a volume of fluidic therapeutic agent conveyed through the fluidic circuit, a pressure of the fluidic therapeutic agent within the fluidic circuit and/or proximal thereto (e.g., at an outlet to fluid conduit 211, a leakage of the fluidic therapeutic agent from the fluidic circuit, a status of power source 216 (e.g., charge status), a device component malfunction, visual images of brain or brain cavity via an implanted imaging device, a detectable signal from the subject, and/or the like. Typically, the detectable signal from the subject is characteristic of at least one neurologically-related disease, condition or disorder. In certain embodiments, the detectable signal from the subject comprises image data. Typically, the fluid and the non-fluid conduits disclosed herein are configured for fluidic, electrical, magnetic, and optical communication between the functional components and the subject. In some embodiments, therapeutic signals include an electrical signal, a magnetic signal, an optical signal, or combinations thereof. In certain embodiments, the functional components are configured to provide acute neurological intervention comprising medicinal therapy, electro-stimulation therapy, radiation therapy, chemotherapy, or a combination thereof. Optionally, one or more of the functional components include, for example, a vital sign monitor, an optical coherence tomography (OCT) image monitor, a high definition camera, an intracranial pressure (ICP) monitor, an electroencephalography sensor (ECOG), a duplex ultrasound monitor, and/or a remote imaging monitor. Additional details regarding other functional components that are optionally adapted for use with the devices disclosed herein are found in, for example, WO 2017/039762 and WO 2018/044984, which are each incorporated by reference in their entirety.

To further illustrate, cranial implant device 200 optionally includes non-fluid-based physiological condition intervention systems that include non-fluid conduit 215 (e.g., a sensor, detector, imaging device, and/or the like). In some embodiments, for example, non-fluid conduit 215 includes an electrode operably connected to cranial implant housing 202, power source 216, and/or controller 214. The electrode is configured to selectively transmit one or more electrical signals to the subject, for example, as part of a course of therapy. In certain embodiments, at least a portion of the electrode is disposed within cranial implant housing 202 and/or extends from second surface 206 of cranial implant housing 202. In other exemplary embodiments, non-fluid conduit 215 includes at least one imaging device (e.g., a visual camera, an ultrasound device (e.g., a duplex ultrasound device), an optical coherence tomography (OCT) device, or the like) operably connected to cranial implant housing 202, power source 216, and/or controller 214. The imaging device is typically configured to selectively capture image data (e.g., low-definition image data and/or high-definition image data) from subjects. Typically, at least a portion of the imaging device is disposed within cranial implant housing 202 and/or at least a portion of the imaging device extends from second surface 206 of cranial implant housing 202.

The functional components include various embodiments. In some embodiments, for example, the functional component include at least one detector that is configured to detect information from the subject and/or the device. To illustrate, the functional component is optionally configured to provide neuron modulation via optic sensors in certain embodiments. In other exemplary embodiments, the functional component is configured for computerized monitoring of at least one physiological condition. Optionally, the functional component includes one or more of an intercranial pressure (ICP) monitor, a vital sign monitor, an imaging device (e.g., a camera, an optical coherence tomography (OCT) device, an ultrasound device, etc.), and the like. To further illustrate, the functional component optionally includes an electrical system, a remote imaging system, a radiation system (e.g., a seed therapy radiation system), a responsive neurostimulation system, and/or a neuromodulation system. Optionally, the functional component includes a medicine delivery device, an electrical signal delivery device, image capture device, radioactive seed device, energy storage device, and/or a computing device. In some embodiments, the functional component includes an electrical energy source, an electrical energy detector, electromagnetic energy source, and/or an electromagnetic energy detector. Typically, the electrical energy source is configured to generate an electrical signal and the electromagnetic energy source is configured to generate an optical signal, and the electromagnetic energy detector is configured to capture image data.

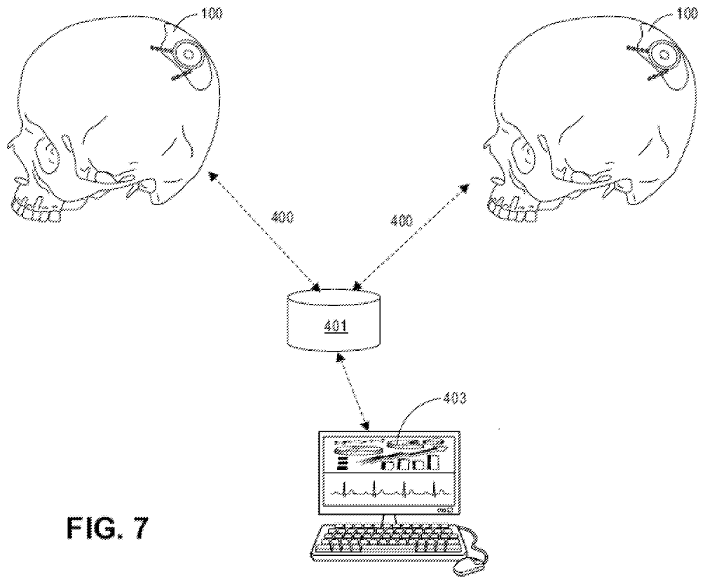

To further illustrate, FIG. 7 schematically illustrates that CED cranial implant devices 100 are optionally provided implanted in multiple subjects with wireless communications capability so as to communicate (as indicated by dashed-lines 400) with a computer 403, via for example, a server 401. In some embodiments, this configuration is used to monitor randomized, controlled clinical trials. While not limited to any particular embodiment, such communication may be via electrical communication (such as via a USB cable) or via electromagnetic communication via Wi-Fi, Bluetooth, or the like. In one example, computer 403 may include a processor that executes software instructions for communicating with the functional component 108 of device 100. As such, remote monitoring of brain activity and/or tumor recurrence reduce healthcare costs associated with hospital-based imaging such as MRI and remove the need to have IVs placed for contrast administration—since the necessary dye are optionally delivered by CED cranial implant devices 100 and imaging is also optionally done remotely by via CED cranial implant devices 100. While not limited to any particular embodiment, computer 403 may be a desktop computer, notebook computer, smart phone, tablet, a virtual reality device, a mixed reality device and server 401 may be a cloud server or another format. Computer 403 may communicate with CED cranial implant devices 100, for example, functional components 108 of CED cranial implant devices 100, via the internet. Functional components 108 may be activated remotely, for example, via signals generated in computer 403. One example is analogous to a 24-hour cardiac heart monitor for which records heart activities for a certain time period. In this case, regrowth of tumor within the cavity would trigger an alarm for notifying the patient and/or healthcare provider. With certain CED cranial implant device embodiments, the implant devices are optionally designed to monitor electrical activity, supranormal intracranial pressures, acute stroke-like bleeding, brain tumor recurrence, or aberrant seizure activity for a certain timeframe, and then at any time, the intervening physician, optionally downloads a recorded database of all activities related to specific intervention (i.e. subclinical seizure activity) that may be visualized on a 2-D and/or 3-D monitor screen. In certain embodiments, computer 403 display data associated with signals generated by the functional component 108 as it monitors patients in whom the device 100 is attached (e.g., to simultaneously monitor courses of treatment for multiple patients, to simultaneously monitor clinical trials in which therapeutic agents are administer to patients via CED cranial implant devices 100, etc.).

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, cranial implant devices, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

What is claimed is:

1. A magnetic resonance imaging (MRI) compatible, convection-enhanced delivery (CED) cranial implant device, comprising:
at least one cranial implant housing configured for intercranial implantation in at least one cranial opening of a subject, which cranial implant housing comprises a substantially anatomically-compatible shape, at least first and second surfaces, and at least one fluidic circuit comprising at least one cavity and at least one port that fluidly communicates with the cavity through at least the second surface, wherein the cavity comprises, or is capable of comprising, at least one fluidic therapeutic agent;
at least one CED pump operably connected to the fluidic circuit, which CED pump is configured to convey the fluidic therapeutic agent from the cavity through at least one fluid conduit when the fluid conduit is operably connected to the port to maintain at least one positive pressure gradient of the fluidic therapeutic agent at least proximal to an outlet of the fluid conduit;
at least one controller operably connected at least to the CED pump, which controller is configured to selectively effect the CED pump to convey the fluidic therapeutic agent through the fluid conduit when the fluid conduit is operably connected to the port and the cavity comprises the fluidic therapeutic agent, and;
at least one power source operably connected at least to the controller,
wherein the cranial implant housing, the CED pump, the controller, and the power source are fabricated from one or more MRI compatible materials.

2. The device of claim 1, wherein the cranial implant housing comprises a standardized form.

3. The device of any one preceding claim, wherein the cranial implant housing comprises a form that is customized for the subject.

4. The device of any one preceding claim, wherein the fluidic therapeutic agent comprises an optogenetic protein, a stem cell, an immune cell, an antibody, an enzyme, a radiation therapeutic agent, a chemical therapeutic agent, a neurological medicine, a neurological preventative medicine, a neurological enhancing medicine, or combinations thereof.

5. The device of any one preceding claim, wherein the fluidic therapeutic agent comprises one or more therapies selected from the group consisting of anti-tumor, anti-seizure, anti-Parkinson, anti-Huntington, anti-hydrocephalus, anti-ADHD, anti-Alzheimer's, anti-pain, anti-insomnia, anti-depression, anti-schizophrenia, energy-enhancing, mind-enhancing, memory-enhancing, neuro-protective, anti-aging, and combinations thereof.

6. The device of any one preceding claim, wherein the fluidic circuit comprises one or more fluidic channels operably connected to the cavity and port.

7. The device of any one preceding claim, wherein the cranial implant housing comprises multiple cavities that each comprise, or are capable of comprising, one or more fluidic therapeutic agents and/or other fluidic materials.

8. The device of any one preceding claim, wherein the cranial implant housing comprises an MRI compatible polymer, an MRI compatible metal, an MRI compatible bioengineered material, or combinations thereof.

9. The device of any one preceding claim, wherein the cranial implant housing comprises one or more of medical-grade titanium, titanium mesh, porous hydroxyapatite (HA), polymethylmethacrylate (PMMA), polyether ether ketone (PEEK), porous polyethylene, cubic zirconia (CZ), or combinations thereof.

10. The device of any one preceding claim, wherein the cranial implant housing comprises a substantially translucent, radiolucent, and/or sonolucent material.

11. The device of any one preceding claim, comprising at least one attachment mechanism or portion thereof operably connected, or connectable, to the cranial implant housing and/or the fluid conduit, which attachment mechanism or portion thereof is configured to attach the fluid conduit to the cranial implant housing such that the fluid conduit fluidly communicates with the fluidic circuit.

12. The device of any one preceding claim, wherein the intercranial implantation is of an indefinite duration.

13. The device of any one preceding claim, comprising multiple ports that fluidly communicate with the cavity through at least the second surface.

14. The device of any one preceding claim, wherein the CED pump, the controller, and the power source are encased within the cranial implant housing.

15. The device of any one preceding claim, wherein the CED pump, the controller, and the power source and optionally one or more other device components are encased within the cranial implant housing to maximize use of dead space between the first and second surfaces.

16. The device of any one preceding claim, wherein the CED pump comprises at least one electroactive polymer (EAP) valve-gated pump.

17. The device of any one preceding claim, wherein the controller is configured for wireless connectivity so as to be remotely monitored, activated, or both.

18. The device of any one preceding claim, wherein the power source comprises at least one battery.

19. The device of any one preceding claim, comprising at least one self-sealing access port disposed at least partially in or through the first surface, which self-sealing access port fluidly communicates with the cavity and is configured to receive one or more syringe needles through the scalp of the subject to add and/or remove the fluidic therapeutic agent to/from the cavity.

20. The device of claim 19, wherein the self-sealing access port comprises a septum.

21. The device of any one preceding claim, comprising one or more detectors at least partially disposed within cranial implant housing and operably connected at least to the controller, which detectors are configured to detect information from the subject and/or the device, which information is selected from the group consisting of: a volume of fluidic therapeutic agent disposed in the cavity, a volume of fluidic therapeutic agent conveyed through the fluidic circuit, a pressure of the fluidic therapeutic agent within the fluidic circuit and/or proximal thereto, a leakage of the fluidic therapeutic agent from the fluidic circuit, a status of the power source, a device component malfunction, visual images of brain or brain cavity via an implanted imaging device, and a detectable signal from the subject.

22. The device of claim 21, wherein the detectable signal from the subject is characteristic of at least one neurologically-related disease, condition or disorder.

23. The device of claim 21, wherein the detectable signal from the subject comprises image data.

24. The device of any one preceding claim, comprising the fluid conduit operably connected to the port.

25. The device of claim 24, wherein the fluid conduit delivers the fluidic therapeutic agent to a diseased portion of brain parenchyma, a dead-space cavity following brain tumor resection, and/or a blood vessel, neuron or ventricle of a brain.

26. The device of claim 24, wherein the fluid conduit comprises a polymer tubing.

27. The device of claim 24, wherein the fluid conduit comprises a catheter.

28. The device of claim 24, wherein the fluid conduit is at least partially disposed within a cannula that is operably connected to the cranial implant housing.

29. The device of claim 24, wherein the second surface of the cranial implant housing comprises 2, 3, 4, or 5 ports that fluidly communicate with one or more fluidic circuits disposed within the cranial implant housing.

30. The device of claim 29, comprising 2, 3, 4, or 5 fluid conduits operably connected to the ports.

31. The device of any one preceding claim, comprising at least one electrode operably connected, or connectable, to the cranial implant housing and/or the controller, which electrode is configured to selectively transmit one or more electrical signals to the subject.

32. The device of claim 31, wherein at least a portion of the electrode is disposed within the cranial implant housing.

33. The device of claim 31, wherein at least a portion of the electrode extends from the second surface of the cranial implant housing.

34. The device of any one preceding claim, comprising at least one imaging device operably connected, or connectable, to the cranial implant housing and/or the controller, which imaging device is configured to selectively capture image data from the subject.

35. The device of claim 34, wherein the imaging device comprises a camera.

36. The device of claim 34, wherein at least a portion of the imaging device is disposed within the cranial implant housing.

37. The device of claim 34, wherein at least a portion of the imaging device extends from the second surface of the cranial implant housing.

38. The device of claim 34, wherein the imaging device comprises an ultrasound device.

39. The device of claim 34, wherein the imaging device comprises an optical coherence tomography (OCT) device.

40. The device of claim 34, wherein the image data comprises low-definition image data.

41. The device of claim 34, wherein the image data comprises high-definition image data.

42. A magnetic resonance imaging (MRI) compatible, convection-enhanced delivery (CED) cranial implant device, comprising:
at least one cranial implant housing configured for intercranial implantation in at least one cranial opening of a subject, which cranial implant housing comprises a substantially anatomically-compatible shape, at least first and second surfaces, and at least one fluidic circuit comprising at least one cavity and at least one port that fluidly communicates with the cavity through at least the second surface, wherein the cavity comprises, or is capable of comprising, at least one fluidic therapeutic agent;
at least one CED pump operably connected to the fluidic circuit, which CED pump is configured to convey the fluidic therapeutic agent from the cavity through at least one fluid conduit when the fluid conduit is operably connected to the port to maintain at least one positive pressure gradient of the fluidic therapeutic agent at least proximal to an outlet of the fluid conduit; and,
at least one power source operably connected at least to the CED pump,
wherein the cranial implant housing, the CED pump, and the power source are fabricated from one or more MRI compatible materials.

43. A cranial implant device, comprising:
at least one cranial implant housing configured for intercranial implantation in at least one cranial opening of a subject;
at least two functional components at least partially disposed within the cranial implant housing, wherein a first functional component comprises a fluid-based physiological condition intervention system that comprises at least one convection-enhanced delivery (CED) pump configured to convey at least one fluidic therapeutic agent from the first functional component to the subject through at least one fluid conduit, and wherein a second functional component comprises a non-fluid-based physiological condition intervention system configured to transmit one or more therapeutic signals from the second functional component to the subject through at least one non-fluid conduit; and,
at least one power source at least partially disposed within the cranial implant housing, which power source is operably connected to the functional components.

44. The device of claim 43, wherein the cranial implant housing, the functional components, and/or the power source are fabricated from one or more magnetic resonance imaging (MRI) compatible materials.

45. The device of any one of claims 43 and 44, wherein the cranial implant housing, the functional components, and/or the power source comprises an MRI compatible polymer, an MRI compatible metal, an MRI compatible bioengineered material, or combinations thereof.

46. The device of any one of claims 43-45, wherein the cranial implant housing, the functional components, and/or the power source comprises one or more of titanium mesh, porous hydroxyapatite (HA), polymethylmethacrylate (PMMA), polyether ether ketone (PEEK), porous polyethylene, cubic zirconia (CZ), or combinations thereof.

47. The device of any one of claims 43-46, wherein the cranial implant housing comprises a substantially anatomically-compatible shape.

48. The device of any one of claims 43-47, wherein the CED pump comprises at least one electroactive polymer (EAP) valve-gated pump.

49. The device of any one of claims 43-48, wherein the power source comprises at least one zero-volt battery.

50. The device of any one of claims 43-49, wherein the cranial implant housing comprises at least first and second surfaces, and at least one fluidic circuit comprising at least one cavity and at least one port that fluidly communicates with the cavity through at least the second surface, wherein the cavity comprises, or is capable of comprising, the fluidic therapeutic agent.

51. The device of claim 50, wherein the CED pump is operably connected to the fluidic circuit.

52. The device of claim 50, wherein the fluidic circuit comprises one or more fluidic channels operably connected to the cavity and port.

53. The device of claim 50, comprising at least one self-sealing access port disposed at least partially in or through the first surface, which self-sealing access port fluidly communicates with the cavity and is configured to receive one or more syringe needles through the scalp of the subject to add and/or remove the fluidic therapeutic agent to/from the cavity.

54. The device of any one of claims 43-53, wherein the functional components are configured to deliver one or more therapies to the subject selected from the group consisting of anti-tumor, anti-seizure, anti-Parkinson, anti-Huntington, anti-hydrocephalus, anti-ADHD, anti-Alzheimer's, anti-pain, anti-insomnia, anti-depression, anti-schizophrenia, anti-aging, energy-enhancing, memory-enhancing, mind-enhancing, neuro-protective, and combinations thereof.

55. The device of any one of claims 43-54, wherein the functional components and the power source are encased within the cranial implant housing.

56. The device of any one of claims 43-55, comprising at least one controller at least partially disposed within the cranial implant housing, which controller is operably connected to the functional components and the power source, and is configured to selectively effect the CED pump of first functional component to convey the fluidic therapeutic agent through the fluid conduit to the subject and the second functional component to transmit the therapeutic signals through the non-fluid conduit to the subject.

57. The device of claim 56, wherein the controller is configured for wireless connectivity so as to be remotely monitored, activated, and/or adjusted.

58. The device of claim 56, wherein the first functional component comprises one or more detectors at least partially disposed within cranial implant housing and operably connected at least to the controller, which detectors are configured to detect information from the subject and/or the device, which information is selected from the group consisting of: a volume of fluidic therapeutic agent disposed in a cavity of the device, a volume of fluidic therapeutic agent conveyed through a fluidic circuit, a pressure of the fluidic therapeutic agent within the fluidic circuit and/or proximal thereto, a leakage of the fluidic therapeutic agent from the fluidic circuit, a status of the power source, a device component malfunction, and a detectable signal from the subject.

59. The device of any one of claims 43-58, wherein the fluid conduit and/or the non-fluid conduit extend from the cranial implant housing.

60. The device of any one of claims 42-58, wherein the fluid conduit and the non-fluid conduit are configured for fluidic, electrical, magnetic, and optical communication between the functional components and the subject.

61. The device of any one of claims 43-60, wherein the therapeutic signals comprise an electrical signal, a magnetic signal, an optical signal, or combinations thereof.

62. The device of any one of claims 43-61, wherein the second functional component comprises at least one detector that is configured to detect information from the subject and/or the device.

63. The device of any one of claims 43-62, wherein the functional components are configured to provide acute neurological intervention comprising medicinal therapy, electro-stimulation therapy, radiation therapy, chemotherapy, or a combination thereof.

64. The device of any one of claims 43-63, wherein one or more of the functional components comprises a vital sign monitor, a brain function monitor, an ultrasound device, an optical coherence tomography (OCT) image monitor, a camera, an intracranial pressure (ICP) monitor, an electroencephalography sensor (ECOG), and/or a remote imaging monitor.

65. The device of any one of claims 43-64, wherein the second functional component is configured to provide neuron modulation via optic sensors.

66. The device of any one of claims 43-65, wherein the second functional component is configured for computerized monitoring of at least one physiological condition.

67. The device of any one of claims 43-66, wherein the second functional component is configured to monitor a diseased portion of brain parenchyma, a dead-space cavity following brain tumor resection, and/or a blood vessel, neuron or ventricle of a brain.

68. The device of any one of claims 43-67, wherein the second functional component comprises at least one intercranial pressure (ICP) monitor.

69. The device of any one of claims 43-68, wherein the second functional component comprises at least one vital sign monitor.

70. The device of any one of claims 43-69, wherein the second functional component comprises at least one imaging device.

71. The device of claim 70, wherein the imaging device comprises a camera.

72. The device of claim 70, wherein the imaging device comprises an optical coherence tomography (OCT) device.

73. The device of claim 70, wherein the imaging device comprises an ultrasound device.

74. The device of any one of claims 43-73, wherein the second functional component comprises an electrical system, a remote imaging system, a radiation system, a responsive neurostimulation system, and/or a neuromodulation system.

75. The device of any one of claims 43-74, wherein the second functional component comprises a medicine delivery device, an electrical signal delivery device, image capture device, radioactive seed device, energy storage device, and/or a computing device.

76. The device of any one of claims 43-75, wherein the second functional component comprises an electrical energy source, an electrical energy detector, electromagnetic energy source, and/or an electromagnetic energy detector.

77. The device of claim 76, wherein the electrical energy source is configured to generate an electrical signal, the electromagnetic energy source is configured to generate an optical signal, and wherein the electromagnetic energy detector is configured to capture image data.

78. A method of treating a neurologically-related disease, condition or disorder of a subject, the method comprising:
surgically implanting at least one cranial implant device in at least one cranial opening of the subject, wherein the cranial implant device comprises:
at least one cranial implant housing that comprises a substantially anatomically-compatible shape, at least first and second surfaces, and at least one fluidic circuit comprising at least one cavity and at least one port that fluidly communicates with the cavity through at least the second surface, wherein the cavity comprises at least one fluidic therapeutic agent, and wherein at least fluid conduit extends from the second surface and fluidly communicates with the fluidic circuit;
at least one convection-enhanced delivery (CED) pump operably connected to the fluidic circuit, which CED pump is configured to convey the fluidic therapeutic agent from the cavity through the fluid conduit to maintain at least one positive pressure gradient of the fluidic therapeutic agent at least proximal to an outlet of the fluid conduit within a cranial cavity of the subject;
at least one controller operably connected at least to the CED pump, which controller is configured to selectively effect the CED pump to convey the fluidic therapeutic agent through the fluid conduit, and;
at least one power source operably connected at least to the controller,
wherein the cranial implant housing, the CED pump, the controller, and the power source are fabricated from one or more magnetic resonance imaging (MRI) compatible materials; and,
conveying an effective amount of the fluidic therapeutic agent from the cavity through the fluid conduit to maintain the positive pressure gradient of the fluidic therapeutic agent at least proximal to the outlet of the fluid conduit within the cranial cavity of the subject, thereby treating the neurologically-related disease, condition or disorder of the subject.

79. The method of claim 78, wherein the neurologically-related disease, condition or disorder comprises one or more of cancer, epilepsy, Parkinson's disease, Huntington's disease, hydrocephalus, attention deficit-hyperactivity disorder (ADHD), pain, Alzheimer's disease, insomnia, depression, manic depression, and schizophrenia.

80. The method of any one of claims 78 and 79, wherein the fluidic therapeutic agent comprises an optogenetic protein, a stem cell, an immune cell, an antibody, an enzyme, a radiation therapeutic agent, a chemical therapeutic agent, a neurological medicine, a neurological preventative medicine, a neuro-enhancing medicine, or combinations thereof.

81. The method of any one of claims 78-80, comprising conveying the effective amount of the fluidic therapeutic agent to a diseased portion of brain parenchyma, a dead-space cavity following brain tumor resection, and/or a blood vessel, neuron or ventricle of the brain of the subject.

82. The method of any one of claims 78-81, wherein at least one self-sealing access port is disposed at least partially in or through the first surface of the cranial implant housing, which self-sealing access port fluidly communicates with the cavity, and the method comprises inserting a syringe needle through the scalp of the subject and through the self-sealing access port, and adding the fluidic therapeutic agent to the cavity.

83. The method of any one of claims 78-82, wherein the controller is configured for wireless connectivity so as to be remotely monitored, activated, or both, and the method comprises wirelessly sending and/or receiving information and/or instructions to/from the controller.

84. The method of any one of claims 78-83, wherein the cranial implant device comprises one or more detectors at least partially disposed within cranial implant housing and operably connected at least to the controller, which detectors are configured to detect information from the subject and/or the device, and wherein the method comprises detecting a volume of fluidic therapeutic agent disposed in the cavity, a volume of fluidic therapeutic agent conveyed through the fluidic circuit, a pressure of the fluidic therapeutic agent within the fluidic circuit and/or proximal thereto, a leakage of the fluidic therapeutic agent from the fluidic circuit, a status of the power source, a device component malfunction, and/or a detectable signal from the subject.

85. The method of any one of claims 78-84, wherein the cranial implant device comprises at least one intercranial pressure (ICP) monitor operably connected to the controller, and wherein the method comprises monitoring the ICP of the subject using the ICP monitor.

86. The method of any one of claims 78-85, wherein the cranial implant device comprises at least one vital sign monitor operably connected to the controller, and wherein the method comprises monitoring one or more vital signs of the subject using the vital sign monitor.

87. The method of any one of claims 78-86, wherein the cranial implant device comprises at least one imaging device operably connected to the controller, and wherein the method comprises capturing image data from the subject using the imaging device.

88. The method of claim 87, wherein the imaging device comprises an optical coherence tomography (OCT) device, and wherein the method comprises capturing OCT image data from the subject using the OCT device.

89. The method of claim 87, wherein the imaging device comprises an ultrasound device, and wherein the method comprises capturing ultrasound image data from the subject using the ultrasound device.

90. The method of any one of claims 78-89, comprising obtaining one or more CT scan and/or MRI images of a cranial cavity of the subject.

91. A method of monitoring therapeutic agent administration in a plurality of subjects, the method comprising:
surgically implanting at least one cranial implant device in each of the plurality subjects, wherein each of the cranial implant devices comprises:
at least one cranial implant housing that comprises a substantially anatomically-compatible shape, at least first and second surfaces, and at least one fluidic circuit comprising at least one cavity and at least one port that fluidly communicates with the cavity through at least the second surface, wherein the cavity comprises at least one fluidic therapeutic agent, and wherein at least fluid conduit extends from the second surface and fluidly communicates with the fluidic circuit;
at least one convection-enhanced delivery (CED) pump operably connected to the fluidic circuit, which CED pump is configured to convey the fluidic therapeutic agent from the cavity through the fluid conduit to maintain at least one positive pressure gradient of the fluidic therapeutic agent at least proximal to an outlet of the fluid conduit within a cranial cavity of a given subject;
at least one controller operably connected at least to the CED pump, which controller is configured to selectively effect the CED pump to convey the fluidic therapeutic agent through the fluid conduit, and for wireless connectivity so as to be remotely monitored, activated, or both, and;
at least one power source operably connected at least to the controller,
wherein the cranial implant housing, the CED pump, the controller, and the power source are fabricated from one or more magnetic resonance imaging (MRI) compatible materials; and,
conveying selected amounts of the fluidic therapeutic agent to one or more members of the plurality of subjects using the implanted cranial implant devices; and,
gathering data from one or more selected sets of members of the plurality of subjects using the wireless connectivity of the implanted cranial implant devices, thereby monitoring the therapeutic agent administration in the plurality of subjects.

92. The method of claim 91, wherein the data correlates with a measure of efficacy and/or toxicity of the therapeutic agent in the plurality of subjects.

93. The method of claim 91, wherein the data correlates with a measure of performance of the cranial implant devices in the plurality of subjects.

94. A surgical method, the method comprising surgically implanting at least one cranial implant device in at least one cranial opening of the subject, wherein the cranial implant device comprises:
at least one cranial implant housing that comprises a substantially anatomically-compatible shape, at least first and second surfaces, and at least one fluidic circuit comprising at least one cavity and at least one port that fluidly communicates with the cavity through at least the second surface, wherein the cavity comprises at least one fluidic therapeutic agent, and wherein at least fluid conduit extends from the second surface and fluidly communicates with the fluidic circuit;
at least one CED pump operably connected to the fluidic circuit, which CED pump is configured to convey the fluidic therapeutic agent from the cavity through the fluid conduit to maintain at least one positive pressure gradient of the fluidic therapeutic agent at least proximal to an outlet of the fluid conduit within a cranial cavity of the subject;
at least one controller operably connected at least to the CED pump, which controller is configured to selectively effect the CED pump to convey the fluidic therapeutic agent through the fluid conduit, and;
at least one power source operably connected at least to the controller,
wherein the cranial implant housing, the CED pump, the controller, and the power source are fabricated from one or more magnetic resonance imaging (MRI) compatible materials.

95. A method of fabricating a cranial implant device, the method comprising:
forming at least first and second portions of a cranial implant housing, wherein once assembled, the first and second portions form at least one cavity and at least one port that fluidly communicates with the cavity through at least one surface of the cranial implant housing to thereby generate at least one fluidic circuit, and wherein the first and second portions are formed from one or more magnetic resonance imaging (MRI) compatible materials;
positioning at least one convection-enhanced delivery (CED) pump relative to the first and/or second portions, wherein the CED pump is formed from one or more MRI compatible materials;
positioning at least one controller relative to the first and/or second portions and operably connecting the controller to the CED pump, wherein the controller is formed from one or more MRI compatible materials;
positioning at least one power source relative to the first and/or second portions and operably connecting the power source to the controller, wherein the power source is formed from one or more MRI compatible materials;
attaching the first and second portions of a cranial implant housing to one another to generate the fluidic circuit and such that the CED pump, the controller, and the power source are encased within the first and second portions, and such that the cranial implant housing comprises a substantially anatomically-compatible shape, thereby fabricating the cranial implant device.

96. An electroactive polymer (EAP) valve-gated pump, comprising:
a top housing structure comprising at least a top surface, wherein at least one top orifice is disposed through the top surface;
a bottom housing structure comprising a substantially concave fluid chamber having a top opening, wherein at least first and second fluid channels fluidly communicate with the fluid chamber;
a membrane portion disposed between the top and bottom housing structures, which membrane portion encloses the concave fluid chamber when the top and bottom housing structures are attached to one another; and,
an EAP-actuation mechanism operably connected to the membrane portion, which EAP-actuation mechanism is configured to displace the membrane portion to thereby effect fluid conveyance.

97. The pump of claim 96, wherein the top and bottom housing structures comprise one or more reversible attachment features configured to reversibly attach the top and bottom housing structures to one another.

98. The pump of claim 96, wherein the membrane portion comprises a silicon membrane.

99. A cranial implant device comprising the pump of claim 96, wherein at least a first fluid conduit is operably connected to the first fluid channel of the bottom housing structure and to a cavity disposed within the cranial implant device, wherein at least a second fluid conduit is operably connected to the second fluid channel of the bottom housing structure and extends from a port disposed through at least one surface of the cranial implant device, and wherein the pump is operably connected to a controller disposed within the cranial implant device.

100. A convection-enhanced delivery (CED) cranial implant device, comprising:
at least one cranial implant housing configured for intercranial implantation in at least one cranial opening of a subject, which cranial implant housing comprises a substantially anatomically-compatible shape, at least first and second surfaces, and at least one fluidic circuit comprising at least one cavity and at least one port that fluidly communicates with the cavity through at least the second surface, wherein the cavity comprises, or is capable of comprising, at least one fluidic therapeutic agent;
at least one CED pump operably connected to the fluidic circuit, which CED pump is configured to convey the fluidic therapeutic agent from the cavity through at least one fluid conduit when the fluid conduit is operably connected to the port to maintain at least one positive pressure gradient of the fluidic therapeutic agent at least proximal to an outlet of the fluid conduit;
at least one controller operably connected at least to the CED pump, which controller is configured to selectively effect the CED pump to convey the fluidic therapeutic agent through the fluid conduit when the fluid conduit is operably connected to the port and the cavity comprises the fluidic therapeutic agent, and;
at least one power source operably connected at least to the controller,
wherein one or more of the cranial implant housing, the CED pump, the controller, the power source, or sub-components thereof, are fabricated from one or more non-MRI compatible materials, which non-MRI compatible materials are selectively and reversibly removable from the CED cranial implant device when the CED cranial implant device is implanted in the subject.

101. A magnetic resonance imaging (MRI) compatible, convection-enhanced delivery (CED) implant device, comprising:
at least one implant housing configured for implantation in at least one opening of a subject, which implant housing comprises a substantially anatomically-compatible shape, at least first and second surfaces, and at least one fluidic circuit comprising at least one cavity and at least one port that fluidly communicates with the cavity through at least the second surface, wherein the cavity comprises, or is capable of comprising, at least one fluidic therapeutic agent;
at least one CED pump operably connected to the fluidic circuit, which CED pump is configured to convey the fluidic therapeutic agent from the cavity through at least one fluid conduit when the fluid conduit is operably connected to the port to maintain at least one positive pressure gradient of the fluidic therapeutic agent at least proximal to an outlet of the fluid conduit;
at least one controller operably connected at least to the CED pump, which controller is configured to selectively effect the CED pump to convey the fluidic therapeutic agent through the fluid conduit when the fluid conduit is operably connected to the port and the cavity comprises the fluidic therapeutic agent, and;
at least one power source operably connected at least to the controller,
wherein the implant housing, the CED pump, the controller, and the power source are fabricated from one or more MRI compatible materials.
